(19) United States
(12) Patent Application Publication
(10) Pub. No.: US 2026/0112502 A1
(43) Pub. Date: Apr. 23, 2026

(54) PLANNING DEVICE, PLANNING METHOD, AND RECORDING MEDIUM

(71) Applicants: NEC Corporation, Tokyo (JP); NATIONAL UNIVERSITY CORPORATION KANAZAWA UNIVERSITY, Kanazawa-shi (JP)

(72) Inventors: Yuki KOSAKA, Tokyo (JP); Yusuke ITO, Tokyo (JP); Shuhei NOYORI, Tokyo (JP); Akihiro NOMURA, Kanazawa-shi (JP); Koki SATSUKAWA, Kanazawa-shi (JP)

(73) Assignees: NEC Corporation, Tokyo (JP); NATIONAL UNIVERSITY CORPORATION KANAZAWA UNIVERSITY, Kanazawa-shi (JP)

(21) Appl. No.: 19/359,845

(22) Filed: Oct. 16, 2025

(30) Foreign Application Priority Data
Oct. 18, 2024 (JP) 2024-184295

(51) Int. Cl. G16H 50/30

(52) U.S. Cl. CPC G16H 50/30

(57) ABSTRACT

A planning device acquires, for each state identified by a value of one or more items correlated with a health-level-evaluation-index value, a feasibility evaluation index value quantitatively indicating feasibility of the state. The planning device acquires a health-level-evaluation-index value for each state. The planning device searches for a path from an initial state to a target state in a plan using an evaluation value for a path involving one or more state transitions. The evaluation value is calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

### CROSS-REFERENCE TO RELATED APPLICATIONS

The present application claims priority to Japanese Patent Application No. 2024-184295, filed Oct. 18, 2024, the contents of which are incorporated herein by reference.

### BACKGROUND

### Technical Field

The present disclosure relates to a planning device, a planning method, and a recording medium.

### Background Art

For the improvement of an individual's health condition, it is conceivable to formulate a plan for gradually changing the combination of quantitative values, such as BMI (Body Mass Index) and blood pressure values. When formulating such a plan, to facilitate its execution, the combination of quantitative values specified in the plan may be designed so as to have high feasibility.

For example, the health improvement path search device disclosed in PCT International Publication No. WO 2022/085785 represents the variable space of multiple explanatory variables, selected from human measurement values obtained through health examinations or the like, as a graph divided into grids, and acquires a health improvement path among the paths connecting the grid points serving as nodes. Specifically, the health improvement path search device identifies paths that transition each measurement target value starting from the current values of multiple explanatory variables, and selects as candidate paths those in which end points have improved health index values compared to their current values. The health improvement path search device then identifies as the health improvement path the path among the candidate paths that maximizes the product of the probability of existence of each measurement target value within the path (the likelihood of existence for each combination of the value of the explanatory variable and the value of the health index).

### SUMMARY

If a subject executing a plan for gradually changing a combination of quantitative values can confirm improvements resulting from the plan, this is expected to provide motivation to continue its execution. If the subject gains motivation to continue executing the plan, the plan is expected to be executed in practice.

An example object of the present disclosure is to provide a planning device, a planning method, and a recording medium capable of solving the problems mentioned above.

According to a first example aspect of the present disclosure, a planning device includes: a memory configured to store instructions; and a processor configured to execute the instructions to: acquire, for each state identified by a value of one or more items correlated with a health-level-evaluation-index value, a feasibility evaluation index value quantitatively indicating feasibility of the state; acquire a health-level-evaluation-index value for each state; and search for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

According to a second example aspect of the present disclosure, a planning method is executed by a computer, and includes: acquiring, for each state identified by a value of one or more items correlated with a health-level-evaluation-index value, a feasibility evaluation index value quantitatively indicating feasibility of the state; acquiring a health-level-evaluation-index value for each state; and searching for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

According to a third example aspect of the present disclosure, a non-transitory computer-readable recording medium stores a program for causing a computer to execute: acquiring, for each state identified by a value of one or more items correlated with a health-level-evaluation-index value, a feasibility evaluation index value quantitatively indicating feasibility of the state; acquiring a health-level-evaluation-index value for each state; and searching for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

According to an example aspect of the present disclosure, it is expected to enable a subject who executes a plan for gradually changing quantitative value combinations to confirm a result of executing the plan.

### BRIEF DESCRIPTION OF THE DRAWINGS

FIG. 1 is a diagram showing a configuration example of a planning device according to at least one of the example embodiments.

FIG. 2 is a diagram showing an example of displaying feasibility-evaluation values on a display unit according to at least one of the example embodiments.

FIG. 3 is a diagram showing an example of displaying health-level-evaluation-index values on the display unit according to at least one of the example embodiments.

FIG. 4 is a diagram showing an example of displaying evaluation index values for each state on the display unit according to at least one of the example embodiments.

FIG. 5 is a diagram showing an example of a model of a feasibility evaluation index used by a feasibility-evaluation-index-value acquisition unit according to at least one of the example embodiments.

FIG. 6 is a diagram showing an example of a processing procedure for the planning device to generate and output a plan, according to at least one of the example embodiments.

FIG. 7 is a diagram showing an example of a processing procedure for a path search unit to perform path searching, according to at least one of the example embodiments.

FIG. 8 is a diagram showing an example of a processing procedure for the path search unit to perform initial setting for path searching, according to at least one of the example embodiments.

FIG. 9 is a diagram showing an example of a processing procedure by which the planning device generates and outputs a plan when a target setting unit sets a target state and then the path search unit performs path searching, according to at least one of the example embodiments.

FIG. 10 is a diagram showing an example of a processing procedure for the planning device to generate and output a plan when a search range is expanded, according to at least one of the example embodiments.

FIG. 11 is a diagram showing an example of the processing procedure for the planning device to generate and output a plan that spans multiple periods, according to at least one of the example embodiments.

FIG. 12 is a diagram showing an example of a feasibility-evaluation-index model used by a feasibility-evaluation-index-value acquisition unit when the planning device generates a plan that spans multiple periods, according to at least one of the example embodiments.

FIG. 13 is a diagram showing an example of displaying a plan that spans multiple periods on the display unit, according to at least one of the example embodiments.

FIG. 14 is a diagram showing a first example of displaying data for each period on the display unit, according to at least one of the example embodiments.

FIG. 15 is a diagram showing a second example of displaying data for each period on the display unit, according to at least one of the example embodiments.

FIG. 16 is a diagram showing a configuration example of the planning device according to at least one of the example embodiments.

FIG. 17 is a diagram showing an example of a processing procedure in a planning method according to at least one of the example embodiments.

FIG. 18 is a diagram showing a configuration example of a computer according to at least one of the example embodiments.

### EXAMPLE EMBODIMENT

Hereinafter, example embodiments of the present disclosure will be described, with reference to the drawings.

In the following, characters with a circumflex may be denoted by appending “\hat{ }” after the character. For example, the character X bearing a circumflex may also be denoted as X\hat{ }.

### First Example Embodiment

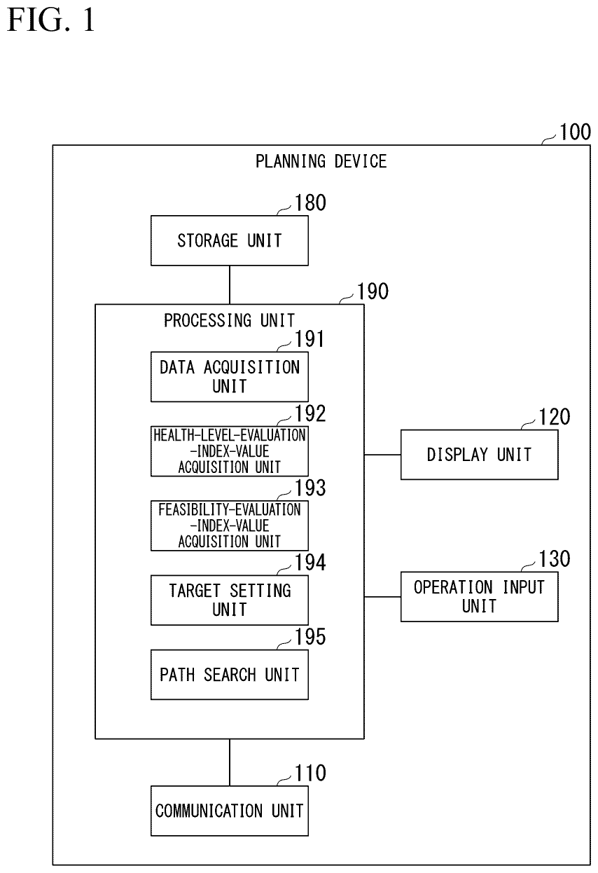

FIG. 1 is a diagram showing a configuration example of a planning device according to at least one of the example embodiments. In the configuration shown in FIG. 1, the planning device 100 includes a communication unit 110, a display unit 120, an operation input unit 130, a storage unit 180, and a processing unit 190.

The processing unit 190 includes a data acquisition unit 191, a health-level-evaluation-index-value acquisition unit 192, a feasibility-evaluation-index-value acquisition unit 193, a target setting unit 194, and a path search unit 195.

The planning device 100 generates a plan for improving health level. Specifically, the planning device 100 formulates a plan for changing values correlated with the health-level-evaluation index, which is an evaluation index of health level, so that the value of the health-level-evaluation-index value indicates a better evaluation. The health level evaluation index can be considered as an index that indicates an evaluation of a certain aspect of health state (the state of the body, mind, or a combination of both).

The planning device 100 may be configured with, for example, a computer such as a PC (Personal computer) or a WS (Workstation).

The plan generated by the planning device 100 can also be referred to as a health promotion plan. The generation of a plan by the planning device 100 can also be considered as plan formulation. The plan generated by the planning device 100 is also simply referred to as a plan.

A healthcare professional, such as the physician responsible for a health checkup, may use the planning device 100 to acquire a plan. Then, the healthcare professional may present the plan generated by the planning device 100 to the subject of the plan (the person who will carry out the plan). Alternatively, the healthcare professional may create a plan by referring to the plan generated by the planning device 100, and present the plan created by the healthcare professional to the subject of the plan.

Alternatively, a person who desires to improve their health state (a subject of a plan) may use the planning device 100 to acquire the plan.

The subject of a plan is also referred to simply as the subject.

The communication unit 110 communicates with other devices. For example, the communication unit 110 may receive data necessary for generating a plan, such as the subject's medical examination results, from another device. Moreover, the communication unit 110 may transmit the plan generated by the planning device 100 to another device.

The display unit 120 includes a display screen such as a liquid crystal panel or an LED (light emitting diode) panel, and displays various types of images. For example, the display unit 120 may display the plan generated by the planning device 100.

The operation input unit 130 includes input devices such as a keyboard and a mouse, and accepts user operations. For example, the operation input unit 130 may receive input operations for various settings related to the generation of a plan, such as a target numerical range of the plan.

The storage unit 180 stores various types of data. For example, the storage unit 180 may store various models such as a health-level-evaluation-index-value model (a model for calculating a health-level-evaluation-index value).

The storage unit 180 is configured using a memory storage device included in the planning device 100.

The processing unit 190 controls each unit of the planning device 100 and executes various processes. Functions of the processing unit 190 are executed by a CPU (central processing unit) included in the planning device 100 reading out a program from the storage unit 180 and executing the program.

The data acquisition unit 191 acquires various data for the planning device 100 to generate a plan. For example, the data acquisition unit 191 may acquire health-checkup-result values of the subject from another device via the communication unit 110.

The health-level-evaluation-index-value acquisition unit 192 acquires the health-level-evaluation-index value of each state within the range set as a target range of the plan. The health-level-evaluation-index-value acquisition unit 192 is an example of the health-level-evaluation-index-value acquisition means.

The health-level-evaluation-index-value acquisition unit 192 may calculate the health-level-evaluation-index value for each state. For example, the health-level-evaluation-index-value acquisition unit 192 may calculate a health-level-evaluation-index value using a health-level-evaluation-index model that has been trained.

Alternatively, the health-level-evaluation-index-value acquisition unit 192 may acquire a health-level-evaluation-index value determined for each state. For example, the health-level-evaluation-index-value acquisition unit 192 may read out a health-level-evaluation value from the storage unit 180, or may acquire the health-level-evaluation-index value from another device via the communication unit 110.

A state (a state in a plan) as referred to herein is a person's health state (a state of the body, mind, or a combination of both). In the plan generated by the planning device 100, discrete states are used, and the states are identified using one or more items correlated with the health-level-evaluation-index value. The items used to identify a state are those whose values are to be directly changed or maintained within the plan. In other words, in the plan, target values of the items used to identify a state are presented, and the subject takes action to achieve those target values. Taking action as referred to herein may also refer to live (daily life).

The items used to identify a state are also referred to as state identifying items. It is preferable that state identifying items are items whose values the subject can relatively easily know, such as items measured in a health checkup, or items whose values the subject can measure or calculate by themselves.

An item that takes a continuous value may be used as a state identifying item, and a divided section of the state identifying item value may be assigned to a state. A representative value, such as the median value of the range assigned to a given state, may be used as the state identifying item value for that state. For example, a representative value assigned to a given state may be used for identifying the state and for calculating a health-level-evaluation-index value or the like in the state.

A state identifying item value used to identify a given state may also be referred to as the state identifying item value for that state.

The processing unit 190 may set the state identifying items. For example, the processing unit 190 may select, as the state identifying items, a predetermined number of items ranked in order of the strongest correlation with the health-level-evaluation-index value from among the measurement target items in the health checkup.

Alternatively, the data acquisition unit 191 may set the state identifying items, such as by reading out the health-level-evaluation-index items and the state identifying items from the storage unit 180.

A range subject to the plan may be set as a predetermined range with an initial state in the plan as a reference. For example, a range subject to the plan may be defined as the range that can be reached from the initial state in the plan within a predetermined number of state transitions. The range subject to the plan (the range set as the target of the plan) is also referred to as the search range.

One state transition can be defined as a transition (movement) from a given state to another adjacent state. Mutually adjacent states may be states in which, for any state identifying item, the assigned sections (numerical ranges) are either adjacent or identical, and yet are not the same state. Alternatively, mutually adjacent states may be states in which, for one of the state identifying items, the assigned sections are adjacent, and for the other state identifying items, the assigned sections are identical.

The processing unit 190 may set the search range. Alternatively, the data acquisition unit 191 may set the search range by, for example, reading out information of the search range from the storage unit 180.

As the initial state in the plan, the state of the subject at the time of plan creation may be used. The initial state in the plan may also be referred to as the starting state in the plan. The initial state in the plan is simply referred to as the initial state or the starting state.

The following explains an example in which the health-level-evaluation index is the onset risk of diabetes, and weight and blood sugar are used as state identifying items.

However, the items targeted by the health-level-evaluation index are not limited to specific items, and may include various items from which the state identifying items can be determined and from which the health-level-evaluation-item value can be measured or calculated. State identifying items are not limited to specific items and may include various items that are correlated with the health-level-evaluation index. The number of state identifying items is not limited to a specific number, and may be one or more.

The onset risk of a disease may represent the probability of developing the target disease within a predetermined period of time. For example, the onset risk of diabetes may be the probability of developing diabetes in the next 10 years, or the probability of developing diabetes in the next 3 years.

The disease targeted by the onset risk is not limited to diabetes. For example, the disease targeted by the onset risk may be a heart disease or a brain disease, but is not limited to these.

For example, the health-level-evaluation-index-value acquisition unit 192 may input health checkup data into a model that predicts onset risk to calculate the onset risk. The model that predicts onset risk is also referred to as the onset risk prediction model, or simply as the prediction model.

The prediction model may be obtained through machine learning. For example, inputting the subject's health checkup data into the prediction model may result in calculating the probability of developing the target disease within three years.

The following describes the prediction model that predicts onset risk. Here, an example of a method for constructing a prediction model that calculates the probability of developing diabetes within three years from a reference year (the year used as the reference for calculating onset risk) is described.

Let the data used to construct the prediction model be (X₁, Y₁), (X₂, Y₂), . . . , (X_{N}, Y_{N}). Each of the data (X₁, Y₁), (X₂, Y₂), . . . , (X_{N}, Y_{N}) is also referred to as training data. The set of data (X₁, Y₁), (X₂, Y₂), . . . , (X_{N}, Y_{N}) is also referred to as training data set.

N is an integer greater than or equal to 1, representing the number of people included in the training data set (the individuals from whom the training data are measured). The people included in the training data set are also referred to as checkup subjects.

The checkup subjects are identified as 1, . . . , N, and the checkup subject from whom the training data (Xₙ, Yₙ) were measured is also denoted as checkup subject n. Here, n is an integer where 1≤n≤N.

Xₙ represents the health checkup data for checkup subject n in the reference year.

Assume that the health checkup data includes data on M health checkup items. M is an integer where M≥1, and indicates the number of health checkup items (number of items).

The health checkup items are also referred to as health checkup item 1, health checkup item 2, . . . , health checkup item M. Assume that among the M health checkup items, there are items that are state identifying items.

Examples of the health checkup items include body weight, height, blood sugar, blood pressure, HDL cholesterol, and LDL cholesterol.

The measurement value of health checkup item j for checkup subject n is also denoted as X_{n,j}. Here, j is an integer where 1≤j≤M.

Assume that none of the N checkup subjects have developed diabetes as of the reference year. Yₙ represents a flag indicating whether or not diabetes developed within three years from the reference year. Yₙ=1 indicates that the disease has developed, and Yₙ=0 indicates that the disease has not developed. The flag indicating whether or not diabetes developed within three years from the reference year is also referred to as the onset flag.

P(Y=1) represents the probability that a person will develop diabetes within three years from the reference year.

The prediction model is constructed using, for example, a training data set {(X₁, Y₁), (X₂, Y₂), . . . , (X_{N}, Y_{N})} for the reference year. Here, consider constructing a prediction model that receives the health checkup data of a subject as input and outputs the probability that the individual will develop diabetes within three years from the reference year.

As a model capable of such input and output, there is a logistic regression model. However, as long as it is a model capable of such input and output, any model other than a logistic regression model may be used.

The following describes a case where a logistic regression model is used.

Let X be an M-dimensional explanatory variable corresponding to the health checkup data in the reference year, and let Y be an objective variable representing whether or not diabetes develops within three years from the reference year.

Let W be an M-dimensional weight vector. The conditional probability P(Y=1|X;W) that Y=1, given the value of X (health checkup data), is shown in Expression (1).

$$\begin{matrix}( Expression 1 ) & \\ P ( Y = 1 \vert X ; W ) = \frac{1}{1 + exp ( {w}^{T} X )} & ( 1 )\end{matrix}$$

A superscript T indicates transposition of a vector or a matrix.

The weight vector W corresponds to the parameter to be adjusted in machine learning.

The conditional probability P(Y=0|X;W) that Y=0 given the value of X is given by Expression (2).

$$\begin{matrix}( Expression 2 ) & \\ P ( Y = 0 \vert X ; W ) = 1 - P ( Y = 1 \vert X ; W ) & ( 2 )\end{matrix}$$

When a training data set {(X₁, Y₁), (X₂, Y₂), . . . , (X_{N}, Y_{N})} is given as data for constructing a predictive model, logistic regression searches for the value of the weight vector W so as to optimize (here, maximize) the value of the objective function shown in Expression (3).

$$\begin{matrix}( Expression 3 ) & \\ L ( W ) = {\sum }_{n = 1}^{N} log P ( {Y}_{n} \vert {X}_{n} ; W ) & ( 3 )\end{matrix}$$

Xₙ and Yₙ respectively indicate the health checkup data and the onset flag value of the checkup subject n.

The value of the objective function L(W) can, for example, be maximized using a method in accordance with the gradient method.

Thus, a method of determining the value of the weight vector W that maximizes the sum of probabilities when the probability P(Yₙ|Xₙ;W) is calculated for the given data (Xₙ, Yₙ)(n=1, . . . , N) is known as the maximum likelihood estimation method.

Here, the value of the weight vector W that maximizes the objective function L(W) is denoted by W*.

Using W* and the values X of M health checkup items of the reference year for the subject (the individual whose risk of developing diabetes is to be predicted), the probability of developing diabetes within three years from the reference year can be calculated using P(Y=1|X;W*). As described above, the M health checkup items include values of state identifying items.

The feasibility-evaluation-index-value acquisition unit 193 acquires the feasibility-evaluation-index value of each state within the search range. The feasibility-evaluation-index-value acquisition unit 193 corresponds to an example of the feasibility-evaluation-index-value acquisition means.

The feasibility evaluation index referred to here is an index that quantitatively represents how easily a state can be realized. For example, in the case where weight and blood sugar are used as state identifying items, the feasibility evaluation value represents how easily a combination of weight and blood sugar values can be realized.

The feasibility-evaluation-index-value acquisition unit 193 may calculate the feasibility-evaluation-index value of each state. For example, the feasibility-evaluation-index-value acquisition unit 193 may calculate a feasibility-evaluation-index value using a trained model of the feasibility evaluation index (a model for calculating the feasibility-evaluation-index value).

Alternatively, the feasibility-evaluation-index-value acquisition unit 193 may acquire a feasibility-evaluation-index value determined for each state. For example, the feasibility-evaluation-index-value acquisition unit 193 may read out the feasibility-evaluation-index value from the storage unit 180, or may acquire the feasibility-evaluation-index value from another device via the communication unit 110.

The feasibility-evaluation-index value may be determined using statistical data. For example, statistical data may be allocated to each state, and the feasibility-evaluation-index value for each state may be calculated so that the greater the number of allocated data, the better the evaluation of the feasibility of that state (the higher the feasibility).

The feasibility-evaluation-index value of a given state may be the occurrence probability of the state (the probability that the state will be realized), but is not limited to this example.

Here, let the number of state identifying items (number of items) be Q, and denote them as state identifying item 1, state identifying item 2, . . . , state identifying item Q. Q is an integer where 1≤Q≤M.

The value of state identifying item q (state identifying item value) is also denoted as X\hat{ }q. Here, q is an integer where 1≤q≤Q.

The state item value X\hat{ } of one person can be represented as a Q-dimensional vector, as shown in Expression (4).

$$\begin{matrix}( Expression 4 ) & \\ \hat{X} = ( {\hat{X}}_{1} , {\hat{X}}_{2} , … , {\hat{X}}_{M} ) & ( 4 )\end{matrix}$$

When distinguishing the state item values of multiple people, they are represented as shown in Expression (5).

$$\begin{matrix}( Expression 5 ) & \\ {\hat{X}}_{n , *} = ( {\hat{X}}_{n , 1} , {\hat{X}}_{n , 2} , … , {\hat{X}}_{n , M} ) & ( 5 )\end{matrix}$$

Here, n is an integer where n≤1, and indicates an index for identifying a person.

In the case where weight and blood sugar are used as state identifying items, the feasibility evaluation value represents how easily a combination of weight and blood sugar values can be realized.

In the following, a method for calculating the occurrence probability of each state is described using, as an example, the case where the state identifying items are body weight and blood sugar. When the state identifying items are weight and blood sugar, the state identifying item value X\hat{ } of one person can be represented by a two-dimensional vector (X\hat{ }₁, X\hat{ }₂). X\hat{ }₁ represents the body weight value. X\hat{ }₂ represents the blood sugar value.

If the types of the possible values (options) for body weight value X\hat{ }₁ are X\hat{ }ⁱ₁ (i=1, . . . , K) and the types of the possible values for blood sugar level X\hat{ }₂ are X\hat{ }^{j}₂ (j=1, . . . , L), then P(XX\hat{ }₁=X\hat{ }ⁱ₁, X\hat{ }₂=X\hat{ }^{j}₂) represents the probability that the body weight value X\hat{ }₁ is X\hat{ }ⁱ₁ and the blood sugar level X\hat{ }₂ is X\hat{ }^{j}₂. K represents the number of types of possible values for the body weight value X\hat{ }₁ (the number of possible values for the body weight value X\hat{ }₁). Moreover, L represents the number of types of possible values for the blood sugar level X\hat{ }₂ (the number of possible values for the blood sugar level X\hat{ }₂).

The probability P(X\hat{ }₁=X\hat{ }ⁱ₁, X\hat{ }₂=X\hat{ }^{j}₂) may be called the occurrence probability.

Expression (6) holds for the probability P(X\hat{ }₁=X\hat{ }ⁱ₁, X\hat{ }₂=X\hat{ }^{j}₂).

$$\begin{matrix}( Expression 6 ) & \\ {\sum }_{i = 1}^{K} {\sum }_{j = 1}^{L} P ( {\hat{X}}_{1} = {\hat{X}}_{1}^{i} , {\hat{X}}_{2} = {\hat{X}}_{2}^{j} ) = 1 & ( 6 )\end{matrix}$$

Table 1 shows an example of the number of people for each combination of weight and blood sugar values when X\hat{ }=(X\hat{ }₁, X\hat{ }₂).

TABLE 1

BODY WEIGHT VALUES: X₁

| | | 60 | 62 | 64 | 66 | TOTAL |
|---|---|---|---|---|---|---|
| BLOOD | 105 | 10 | 2 | 21 | 13 | 46 |
| SUGAR | 110 | 8 | 10 | 22 | 18 | 58 |
| VALUES: | 115 | 0 | 27 | 7 | 5 | 39 |
| X₂ | 120 | 2 | 11 | 39 | 16 | 68 |
| | TOTAL | 20 | 50 | 89 | 52 | 211 |

The value of each cell in Table 1 indicates the number of people whose body weight value is X\hat{ }ⁱ₁ and whose blood sugar level is X\hat{ }^{j}₂.

Table 1 shows an example of a result of allocating 211 individuals corresponding to combinations of possible values, in a case where possible values that a body weight value X₁ can take are 60, 62, 64, and 66, and possible values that a blood sugar value X₂ can take are 105, 110, 115, and 120.

In the example of Table 1, K=4 and L=4. Also, N=211.

Table 2 shows the probabilities in the case of Table 1, where the body weight value X\hat{ }₁ takes X\hat{ }ⁱ₁ and the blood sugar value X\hat{ }₂ takes X\hat{ }^{j}₂.

TABLE 2

| | | BODY WEIGHT VALUES: X₁ | | | | |
|---|---|---|---|---|---|---|
| | | 60 | 62 | 64 | 66 | TOTAL |
| BLOOD | 105 | 0.05 | 0.01 | 0.10 | 0.06 | 0.22 |
| SUGAR | 110 | 0.04 | 0.05 | 0.10 | 0.09 | 0.27 |
| VALUES: | 115 | 0.00 | 0.13 | 0.03 | 0.02 | 0.18 |
| X₂ | 120 | 0.01 | 0.05 | 0.18 | 0.08 | 0.32 |
| | TOTAL | 0.09 | 0.24 | 0.42 | 0.25 | 1.00 |

The probability shown in each cell of Table 2 can be calculated by dividing the number of people whose body weight value is X\hat{ }ⁱ₁ and whose blood sugar level is X\hat{ }^{j}₂ shown in each cell of Table 1 by the total number of people N.

For the probabilities shown in Table 2, Expression (7) holds.

$$\begin{matrix}( Expression 7 ) & \\ {\sum }_{i = 1}^{4} {\sum }_{j = 1}^{4} P ( {\hat{X}}_{1} = {\hat{X}}_{1}^{i} , {\hat{X}}_{2} = {\hat{X}}_{2}^{j} ) = 1 & ( 7 )\end{matrix}$$

Here, the case where the state identifying item value takes a discrete value has been described as an example, but an occurrence probability can be similarly calculated in a case where the state identifying item value takes a continuous value.

The health-level-evaluation-index value may also be determined using statistical data. For example, statistical data may be allocated to each state, and a health-level-evaluation-index value of each state may be determined based on a state regarding an item related to a health-level-evaluation index in the allocated data. In the case where the health-level-evaluation index is the onset risk of diabetes and the state identifying items are body weight and blood sugar, the data can be allocated to each state based on body weight and blood sugar values, and the proportion of data items that show diabetes (the number of people who have developed diabetes) among the number of data items assigned to a certain state (the number of people assigned to a certain state) can be used as the health-level-evaluation-index value for that state.

However, the method for determining the health-level-evaluation-index value is not limited to a specific method.

When the planning device 100 generates a plan, the feasibility-evaluation-index-value acquisition unit 193 may acquire the feasibility-evaluation-index value of the state required for generating the plan. The feasibility-evaluation-index value is not necessarily required to determine the target state in the plan. From this, it is not always necessary for the feasibility-evaluation-index-value acquisition unit 193 to acquire the feasibility-evaluation-index value for every state within the search range.

The target state in the plan is also simply referred to as the target state.

The target setting unit 194 determines the target state based on the health-level-evaluation-index value acquired for each state by the health-level-evaluation-index-value acquisition unit 192. The target setting unit 194 corresponds to an example of the target state setting means. For example, the target setting unit 194 may select, as the target state, the state that indicates the best evaluation of the health-level-evaluation-index value among the states within the search range.

The path search unit 195 searches for a path from the initial state to the target state. A path from the initial state to the target state can be considered as a plan to improve health level. Path searching performed by the path search unit 195 can be considered as generating a plan.

The path search unit 195 corresponds to an example of the path search means.

The path search unit 195 searches for a path from the initial state to the target state using an evaluation value for a path involving one or more state transitions, which is calculated using an evaluation value for a single state transition.

Here, evaluation values are used, each of which indicates, as an evaluation for a single state transition, a better evaluation where the transition-destination state has higher feasibility and also indicates a better evaluation where the health-level-evaluation-index value in the transition-destination state shows a better evaluation relative to the health-level-evaluation-index value in the transition-source state.

By having the evaluation value for a single state transition indicate a better evaluation with higher feasibility of the transition-destination state, the path search unit 195 is expected to more easily select a path that follows comparatively feasible states. That is to say, it is expected that the path search unit 195 will generate a plan indicating intermediate values (intermediate target values) and target values (final target values) of the state identifying items that are comparatively feasible for the subject to achieve.

Moreover, by having the evaluation value for a single state transition indicate a better evaluation as the health-level-evaluation-index value in the transition-destination state indicates a better evaluation compared to that in the transition-source state, the path search unit 195 is expected to generate a plan that enables the subject to confirm progress at each state transition.

It is expected that the ability to confirm progress through the plan generated by the path search unit 195 will serve as motivation for the subject to continue executing the plan. It is expected that the motivation gained to continue executing the plan will enable the subject to execute it through to completion and achieve the outcomes intended in the plain.

The path search unit 195 may use evaluation values for individual state transitions, each of which is determined as a weighted sum of an evaluation value indicating, as an evaluation for a single state transition, a better evaluation where the transition-destination state has higher feasibility, and an evaluation value indicating a better evaluation where the health-level-evaluation-index value in the transition-destination state shows a better evaluation relative to the health-level-evaluation-index value in the transition-source state.

In such a case, by adjusting the weights, the path search unit 195 can adjust the degree to which it generates a plan that indicates intermediate values (intermediate target values) and target values (final target values) of state identifying items that are comparatively more feasible for the subject, and generates a plan that allows the subject to confirm the results for each state transition.

The evaluation value that indicates a better evaluation where the transition-destination state has higher feasibility is also referred to as a feasibility evaluation value. An evaluation value that indicates a better evaluation where the health-level-evaluation-index value in the transition-destination state indicates a better evaluation relative to the health-level-evaluation-index value in the transition-source state is also referred to as a result-confirmability evaluation value.

The path search unit 195 may use the health-level-evaluation-index value in the transition-destination state as an evaluation value that indicates a better evaluation where the health-level-evaluation-index value in the transition-destination state indicates a better evaluation relative to the health-level-evaluation-index value in the transition-source state. For transitions from the same transition-source state to each of its adjacent states, the ranking of evaluations among the respective transition-destination states remains the same, whether an evaluation value that indicates a better evaluation where the health-level-evaluation-index value in the transition-destination indicates a better evaluation relative to that in the transition-source state, is used, or a health-level-evaluation-index value in the transition-destination is used.

The path search unit 195 may search for a path from the initial state to the target state after the target setting unit 194 sets a target state. In such a case, once a path from the initial state to the target state is detected, the path search unit 195 can end the processing, and there is no need to search for further paths to other states. In this respect, it is expected that the amount of calculation required by the path search unit 195 can be made relatively small.

Alternatively, the path search unit 195 may search for a path from the initial state to each state within the search range, and then the target setting unit 194 may set the target state. In such a case, the path search unit 195 can search for a path in advance (before the target setting unit 194 sets the target state). In this respect, the planning device 100 can generate a plan in real time from the setting of a target state.

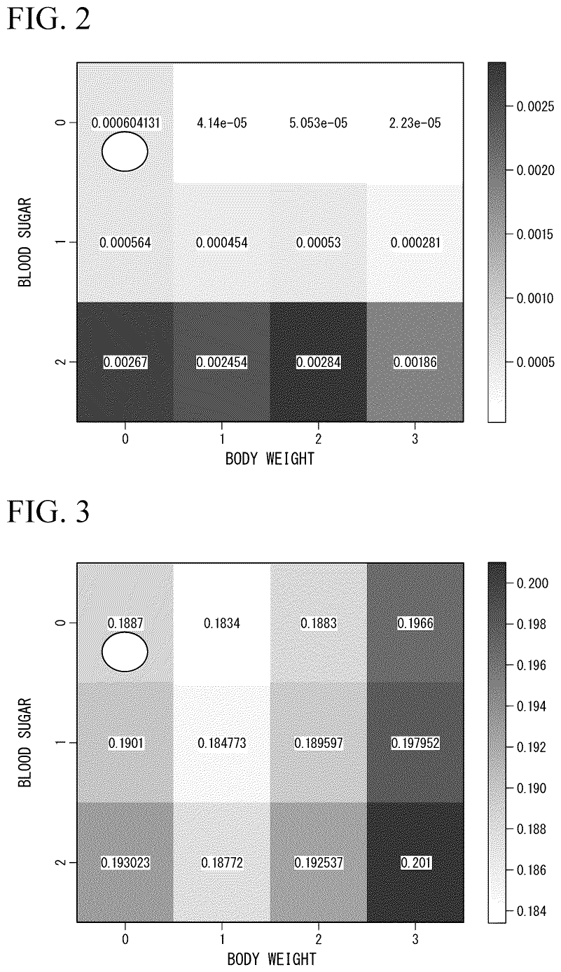

FIG. 2 is a diagram showing an example of feasibility evaluation values displayed on the display unit 120.

FIG. 2 shows a case, as an example, where the state identifying items are weight and blood sugar, with the horizontal direction (horizontal direction of the figure) corresponding to the weight values and the vertical direction (vertical direction of the figure) corresponding to the blood sugar values.

Moreover, along the horizontal direction, column numbers are shown as 0, 1, 2, and 3, increasing from left to right. A smaller column number (and therefore a position further to the left in FIG. 2) indicates a higher body weight. Along the vertical direction, row numbers are shown as 0, 1, and 2, increasing from top to bottom. A smaller row number (and therefore a position higher up in FIG. 2) indicates a higher blood sugar value.

The display unit 120 may show representative values or sections for body weight in each column, and representative values or sections for blood sugar in each row.

Also, in the example of FIG. 2, as a feasibility evaluation value, an occurrence probability of a combination of a body weight value and a blood sugar value assigned to each state is shown. In such a case, the higher the feasibility-evaluation-index value, the higher the feasibility of the state (the state indicated by that feasibility-evaluation-index value) for the subject.

Also, in the example of FIG. 2, a state at row 0 and column 0 (the leftmost and uppermost state in FIG. 2) is an initial state.

FIG. 3 is a diagram showing an example of health-level-evaluation-index values displayed on the display unit 120.

FIG. 3 shows a case, as an example, where the state identifying items are weight and blood sugar, with the horizontal direction (horizontal direction of the figure) corresponding to the weight values and the vertical direction (vertical direction of the figure) corresponding to the blood sugar values.

Moreover, along the horizontal direction, column numbers are shown as 0, 1, 2, and 3, increasing from left to right. A smaller column number (and therefore a position further to the left in FIG. 2) indicates a higher body weight. Along the vertical direction, row numbers are shown as 0, 1, and 2, increasing from top to bottom. A smaller row number (and therefore a position higher up in FIG. 2) indicates a higher blood sugar value.

The display unit 120 may show representative values or sections for body weight in each column, and representative values or sections for blood sugar in each row.

In the example of FIG. 3, 1-onset risk (the value obtained by subtracting the onset risk of diabetes from 1) is shown as the health-level-evaluation-index value. In such a case, the higher the health-level-evaluation-index value, the better the subject's health state.

Also, in the example of FIG. 3, a state at row 0 and column 0 (the leftmost and uppermost state in FIG. 3) is an initial state.

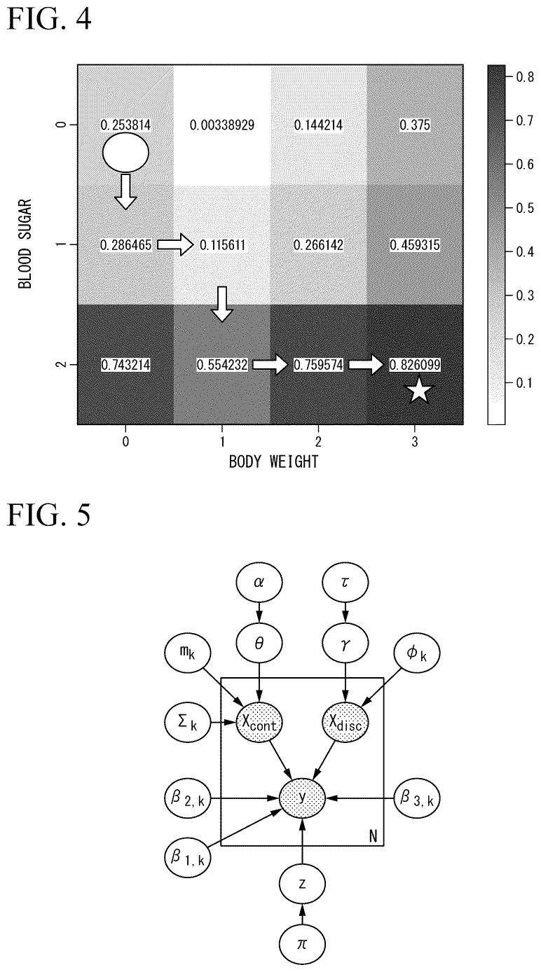

FIG. 4 is a diagram showing an example of evaluation index values displayed on the display unit 120.

FIG. 4 shows a case, as an example, where the state identifying items are weight and blood sugar, with the horizontal direction (horizontal direction of the figure) corresponding to the weight values and the vertical direction (vertical direction of the figure) corresponding to the blood sugar values.

Moreover, along the horizontal direction, column numbers are shown as 0, 1, 2, and 3, increasing from left to right. A smaller column number (and therefore a position further to the left in FIG. 2) indicates a higher body weight. Along the vertical direction, row numbers are shown as 0, 1, and 2, increasing from top to bottom. A smaller row number (and therefore a position higher up in FIG. 2) indicates a higher blood sugar value.

The display unit 120 may show representative values or sections for body weight in each column, and representative values or sections for blood sugar in each row.

Also, in the example of FIG. 4, as an evaluation index value for each state, an occurrence probability+1-diabetes onset risk (a sum of an occurrence probability of a combination of a body weight value and a blood sugar value assigned to each state, and a value obtained by subtracting the diabetes onset risk from 1) is shown.

Also, in the example of FIG. 4, a state at row 0 and column 0 (the leftmost and uppermost state in FIG. 4) is an initial state. A state at row 2 and column 3 (the rightmost and lowermost state in FIG. 4) is an initial state.

The path search unit 195 may use a value obtained by subtracting the evaluation index value for each state at the transition source from the evaluation index value for each state at the transition destination as the evaluation value for one state transition. Then, the path search unit 195 may select a path in which there are many transitions in which the evaluation value for one state transition is a positive value, and in which the variation between transitions in the evaluation value for one state transition is small.

For example, the path search unit 195 may search for a path that maximizes the value of the evaluation function f shown in Expression (8).

$$\begin{matrix}( Expression 8 ) & \\ f = \sum log ( ReLU ( {x}_{i} ) - 2 \times ReLU ( - {x}_{i} ) + 5 ) & ( 8 )\end{matrix}$$

xᵢ indicates the evaluation value for the i-th state transition on a path (evaluation value for one state transition). As xᵢ, a value obtained by subtracting the evaluation index value for each state at the transition source from the evaluation index value for each state at the transition destination of the i-th state transition can be used.

ReLU stands for Rectified Linear Unit and is expressed as in Expression (9).

$$\begin{matrix}( Expression 9 ) & \\ ReLU ( x ) = \{ \begin{matrix}x & when x \geq 0 \\ 0 & when x < 0\end{matrix} & ( 9 )\end{matrix}$$

The base of the logarithm (log) here is not limited to a specific value. For example, in Expression (8), either a common logarithm or a natural logarithm may be used as log.

log(ReLU(xᵢ)−2×ReLU(−xᵢ)+5) corresponds to an example of an evaluation value for a single state transition.

The value of ReLU(xᵢ)−2×ReLU(−xᵢ) in Expression (8) is xᵢ if xᵢ≥0, and is 2xᵢ if xᵢ<0. Thus, if xᵢ≥0, ReLU(xᵢ)−2×ReLU(−xᵢ) takes a positive value. If xᵢ<0, ReLU(xᵢ)−2×ReLU(−xᵢ) takes a negative value whose magnitude (absolute value) is greater than when xᵢ≥0 due to the multiplication of ReLU(−xᵢ) by the coefficient −2.

Accordingly, in a case of a state transition in which a health-level-evaluation-index value decreases due to the state transition, a decrease width of a value of the evaluation function f becomes large, and it is expected that the path search unit 195 selects a path in which the number of state transitions in which the health-level-evaluation-index value decreases is relatively small. In the plan, when the number of state transitions in which the health-level-evaluation-index value decreases is small, the subject has many opportunities to confirm, during the execution of the plan, that the health-level-evaluation-index value has not decreased (that is to say, the health state has not deteriorated). This is expected to improve the subject's motivation to continue executing the plan.

The “+5” in Expression (8) ensures that the value of log(ReLU(xᵢ)−2×ReLU(−xᵢ)+5) is positive. Since the value of log(ReLU(xᵢ)−2×ReLU(−xᵢ)+5) is positive, the path search unit 195 can use a path search algorithm that requires the graph's edge weights to be zero or greater.

However, the “+5” in Expression (8) is just an example, and various values can be added depending on the form of the expression and the values that xᵢ can take. Moreover, a value greater than +5 may be added in Expression (8).

In Expression (8), “+5” is set assuming that both the feasibility-evaluation-index value and the health-level-evaluation-index value take values in the range from 0 to 1, and −4≤ReLU(xᵢ)−2×ReLU(−xᵢ)≤2. As in the above example, in the case where the occurrence probability is used as the feasibility-evaluation-index value, the feasibility-evaluation-index value takes a value in the range from 0 to 1. As in the above example, in the case where the onset risk is used as the health-level-evaluation-index value, the health-level-evaluation-index value takes a value in the range from 0 to 1.

Alternatively, the path search unit 195 may pre-calculate an evaluation index value for each state before starting path searching, and determine a value to be added to ReLU(xᵢ)−2×ReLU(−xᵢ).

Moreover, by taking the logarithm (log) of (ReLU(xᵢ)−2×ReLU(−xᵢ)+5) in Expression (8), it is expected that the increase in the value of the evaluation function f will be suppressed when the increase in the width of the health-level-evaluation-index value during a single state transition is large (the increase in the width of the evaluation function f will be smaller than when the logarithm is not taken). This is expected to enable the path search unit 195 to detect a path in which the health-level-evaluation-index value gradually increases with each state transition (rather than a path in which the health-level-evaluation-index value increases significantly with a singlestate transition). As the health-level-evaluation-index value gradually increases with each state transition, the subject can confirm that the health-level-evaluation-index value is increasing (that is to say, their health state is improving) during the course of the plan execution. This is expected to improve the subject's motivation to continue executing the plan.

Here, consideration is given to a comparison between the case where the path search unit 195 uses an evaluation function f that takes a logarithm (log), as in Expression (8), and the case where it uses an evaluation function f that does not take a logarithm.

In a case where the path search unit 195 uses an evaluation function f that does not take a logarithm, there is a possibility that the path search unit 195 may derive a path where the evaluation function f takes a large value at just a few points, while taking a small value everywhere else.

If the path search unit 195 were to derive such a path, the subject would frequently be unable to confirm an increase in their health-level-evaluation-index value with each transition during the plan's execution. Consequently, the subject's motivation to continue with the plan's execution may not improve and could even decrease.

In contrast, when the path search unit 195 uses an evaluation function f with a logarithm as shown in Expression (8), the resulting path is expected to have many transitions where the subject can confirm an increase in their health-level-evaluation-index value. This is expected to improve the subject's motivation to continue executing the plan.

In the example shown in FIG. 4, the display unit 120 shows the path found by the path search unit 195 by using arrows to indicate the individual state transitions.

However, the evaluation function used by the path search unit 195 for path searching is not limited to any specific one. In particular, the evaluation function used by the path search unit 195 to search for a path is not limited to one that takes a logarithm. For example, the path search unit 195 may search for a path that maximizes the value of the evaluation function f shown in Expression (10).

$$\begin{matrix}( Expression 10 ) & \\ f = \sum ( ReLU ( {x}_{i} ) - 2 \times ReLU ( - {x}_{i} ) + 4 ) & ( 10 )\end{matrix}$$

ReLU(xᵢ)−2×ReLU(−xᵢ)+4 corresponds to an example of an evaluation value for one state transition.

The evaluation function f shown in Expression (10) corresponds to an example of an evaluation function calculated by a method other than the logarithm method. Specifically, the evaluation function f shown in Expression (10) corresponds to an example in which the calculation of taking the logarithm is removed from the evaluation function f shown in Expression (8).

According to the evaluation function f shown in Expression (10), as described for the sub-formula ReLU(xᵢ)−2×ReLU(−xᵢ) of Expression (8), in the case of a state transition that reduces the health-level-evaluation-index value, the degree of reduction in the value of the evaluation function f becomes large, and it is expected that the path search unit 195 will select a path with a relatively small number of state transitions that reduce the health-level-evaluation-index value. In the plan, when the number of state transitions in which the health-level-evaluation-index value decreases is small, the subject has many opportunities to confirm, during the execution of the plan, that the health-level-evaluation-index value has not decreased (that is to say, the health state has not deteriorated). This is expected to improve the subject's motivation to continue executing the plan.

It should be noted that the “+4” in Expression (10) is just an example, and various values can be added depending on the form of the expression and the values that xᵢ can take. Moreover, a value greater than +4 may be added in Expression (10).

Also, the path search unit 195 may pre-calculate an evaluation index value for each state before starting path searching, and determine a value to be added to ReLU(xᵢ)−2×ReLU(−xᵢ).

Alternatively, the path search unit 195 may search for a path that maximizes the value of the evaluation function f shown in Expression (11).

$$\begin{matrix}( Expression 11 ) & \\ f = \sum log ( {x}_{i} + 3 ) & ( 11 )\end{matrix}$$

log(xᵢ+3) corresponds to an example of an evaluation value for one state transition.

The evaluation function f shown in Expression (11) corresponds to another example of an evaluation function using the logarithm method. Specifically, the evaluation function f shown in Expression (11) corresponds to an example in which the calculation using ReLU is removed from the evaluation function f shown in Expression (8).

In such a case, as described with reference to Expression (8), if the increase in the width of the health-level-evaluation-index value during one state transition is large, it is expected that the increase in the width of the value of the evaluation function f will be suppressed (the increase in the width of the evaluation function f will be smaller than when the logarithm is not taken). This is expected to enable the path search unit 195 to detect a path in which the health-level-evaluation-index value gradually increases with each state transition (rather than a path in which the health-level-evaluation-index value increases significantly with a single state transition). As the health-level-evaluation-index value gradually increases with each state transition, the subject can confirm that the health-level-evaluation-index value is increasing (that is to say, their health state is improving) during the course of the plan execution. This is expected to improve the subject's motivation to continue executing the plan.

It should be noted that the “+3” in Expression (11) is just an example, and various values can be added depending on the form of the expression and the values that xᵢ can take. Moreover, a value greater than +3 may be added in Expression (11).

Furthermore, the path search unit 195 may pre-calculate an evaluation index value for each state before starting path searching and determine the value to be added to xᵢ.

Also, in the case where the path search unit 195 performs path searching using Dijkstra's Algorithm, in Dijkstra's Algorithm, the edge weights of the graph must be 0 or more, and furthermore, the weights need to represent costs. In other words, Dijkstra's algorithm uses weights with values of 0 or more to search for a path that minimizes the cumulative value of the weights.

In the case where the path search unit 195 performs path searching using the Dijkstra algorithm and uses the natural logarithm as log, Expression (8) may be transformed into Expression (12) so that the value of the evaluation function f represents the cost.

$$\begin{matrix}( Expression 12 ) & \\ f = \sum ( 2 - log ( ReLU ( {x}_{i} ) - 2 \times ReLU ( - {x}_{i} ) + 5 ) ) & ( 12 )\end{matrix}$$

If −2≤xᵢ≤2, then 1≤ReLU(xᵢ)−2×ReLU(−xᵢ)+5≤7, and therefore 2-log(ReLU(xᵢ)−2×ReLU(−xᵢ)+5)≥0. Furthermore, the larger the value of xᵢ (and therefore the better the evaluation), the smaller the value of the evaluation function f. 2-log(ReLU(xᵢ)−2×ReLU(−xᵢ)+5) corresponds to an example of an evaluation value for a single state transition.

FIG. 5 is a diagram showing an example of a model representing the health-level-evaluation-index value and the feasibility-evaluation-index value.

In other words, the model shown in FIG. 5 may be used as a model for the health-level-evaluation-index-value acquisition unit 192 to calculate the health-level-evaluation-index value. Moreover, the feasibility-evaluation-index-value acquisition unit 193 may use the model shown in FIG. 5 as a model for calculating the feasibility-evaluation-index value.

FIG. 5 shows a case, as an example, where the health-level-evaluation-index value and the feasibility-evaluation-index value are expressed using a hierarchical Bayesian model.

N represents the number of people undergoing health checkup. The “N” in FIG. 5 indicates that a model is constructed using a training data set of N subjects.

y is a variable indicating the health-level-evaluation-index value.

For example, the flag Yₙ mentioned above, which indicates whether or not diabetes developed within three years from the reference year, can be considered to indicate the correct value of the variable y in the training data.

As described above regarding the calculation of diabetes onset risk, the training data for the reference year were expressed as (Xₙ, Yₙ) (n=1, . . . , N). An example was then given in which these pairs (Xₙ, Yₙ) were used to construct a prediction model for the health-level-evaluation index.

In the description of constructing a prediction model using a logistic regression model, X is an M-dimensional explanatory variables corresponding to the health checkup data in the reference year, and Y is an objective variable representing whether or not diabetes develops within three years from the reference year.

X_{cont} or X_{disc}, or a combination thereof, corresponds to the variables for calculating the feasibility-evaluation-index value.

X_{cont} indicates explanatory variables that take continuous values (parameters that take values that follow a continuous distribution) among the M-dimensional explanatory variables corresponding to the health checkup data for the reference year. Body weight and blood sugar are examples of test values that follow a continuous distribution.

In relation to X_{cont}, k indicates an index that identifies each individual continuous distribution constituting a mixed distribution. Here, a Gaussian mixture distribution is used as a continuous distribution. mₖ indicates the mean value in each Gaussian distribution. Σₖ indicates the variance-covariance matrix in each Gaussian distribution.

The combination of α and θ indicates the parameters of the Gaussian mixture distribution. α and θ correspond to examples of latent variables.

θ represents a K-dimensional vector, and the k-th dimension value is θₖ∈{0, 1}. θₖ=1 is an indicator that refers to the k-th Gaussian mixture distribution. Also, Σₖ₌₁^{K}θₖ=1. Here, k is an integer where 1≤k≤K.

α represents the mixture probability (weight for each Gaussian distribution).

Also, αₖ represents the probability that θₖ equals 1. That is, P(θₖ=−1)=αₖ. Here, 0≤αₖ≤1.

Rewriting P(θₖ=1)=αₖ with θₖ as a probability, it can be expressed as in Expression (13).

$$\begin{matrix}( Expression 13 ) & \\ P ( {θ}_{k} = 1 ) = {\prod }_{k = 1}^{K} {α}_{k}^{{θ}_{k}} & ( 13 )\end{matrix}$$

The conditional probability of X, given the K-th Gaussian distribution, can be expressed as in Expression (14).

$$\begin{matrix}( Expression 14 ) & \\ P ( X \vert {θ}_{k} = 1 ) = {N ( X \vert {m}_{k} , {Σ}_{k} )}^{{θ}_{k}} & ( 14 )\end{matrix}$$

Using the vector θ, it can be expressed as in Expression (15).

$$\begin{matrix}( Expression 15 ) & \\ P ( X \vert θ ) = {\prod }_{k = 1}^{K} {N ( X \vert {m}_{k} , {Σ}_{k} )}^{{θ}_{k}} & ( 15 )\end{matrix}$$

Moreover, the marginal distribution P(X) of X can be expressed as in Expression (16) using P(X|θ)P(θ).

$$\begin{matrix}( Expression 16 ) & \\ P ( X ) = P ( X \vert θ ) P ( θ ) & ( 16 )\end{matrix}$$

P(X) can be expressed as in Expression (17).

$$\begin{matrix}( Expression 17 ) & \\ P ( X ) = {\prod }_{k = 1}^{K} {α}_{k}^{{θ}_{k}} {N ( X \vert {m}_{k} , {Σ}_{k} )}^{{θ}_{k}} & ( 17 )\end{matrix}$$

X_{disc} indicates parameters, among the parameters used for calculating the health-level-evaluation-index value and the feasibility-evaluation-index value, that take discrete values (parameters that follow a discrete distribution). Gender and responses to questionnaire items in health checkups correspond to examples of parameters following discrete distributions. In relation to X_{disc}, k indicates an index that identifies each individual discrete distribution constituting a mixed distribution. Here, a categorical distribution is used as the discrete distribution. (k indicates the parameter for the k-th categorical distribution. The parameters of the K categorical distributions are expressed as φ=(φ₁, . . . , φₖ, . . . , φ_{K}).

Here, if X is data on a checkup subject who has explanatory variables that take continuous values among the M-dimensional explanatory variables corresponding to the health checkup data for the reference year, then P(X) represents the occurrence probability of X. This occurrence probability P(X) may be treated in the same manner as the probability P(X\hat{ }₁=X\hat{ }ⁱ₁, X\hat{ }₂=X\hat{ }^{j}₂) in Expression (6), and P(X) may be used as a feasibility-evaluation-index value.

The combination of τ and γ indicates the parameters of the mixed categorical distribution. τ and γ correspond to examples of latent variables. The combination of τ and γ is similar to the combination of the parameters α and θ of the Gaussian mixture distribution.
γ represents a K-dimensional vector, and the value of the k-th dimension is γₖ={0, 1}. γₖ=1 is an indicator that refers to the k-th Gaussian mixture distribution. Also, Σₖ=1^{K}γₖ=1.
τ represents the mixture probability (weight for the Gaussian distribution from each category).

As mentioned above, when using logistic regression, the conditional probability P(Y=1|X;W) that Y equals 1 is expressed as in Expression (1) above. This conditional probability P may be treated in the same manner as the probability P(Y=1 X;W*) mentioned above, and the conditional probability P may be used as a health-level-evaluation-index value.

W^{T}X indicates the sum, across the M dimensions, of the products of the elements of the weight vector W and the elements of the explanatory variable vector X, as expressed in Expression (18).

$$\begin{matrix}( Expression 18 ) & \\ {W}^{T} X = {W}_{1} {X}_{1} + {W}_{2} {X}_{2} + … + {W}_{M} {X}_{M} & ( 18 )\end{matrix}$$

In FIG. 5, the weight vector W is represented by β.

FIG. 5 also shows an example where M=3.

Moreover, in FIG. 5, P(Y=1|X;W) can be considered to be represented by a mixed logistic regression model.

k indicates an index that identifies the individual logistic regressions that make up the mixture distribution. βₖ indicates the parameter of the k-th logistic regression.

In FIG. 5, βₖ=(β_{1, k}, β_{2, k}, β_{3, k}).

The combination of π and z represents the parameters of the mixed logistic regression. The combination of π and z is similar to the combination of the parameters a and θ of the Gaussian mixture distribution.

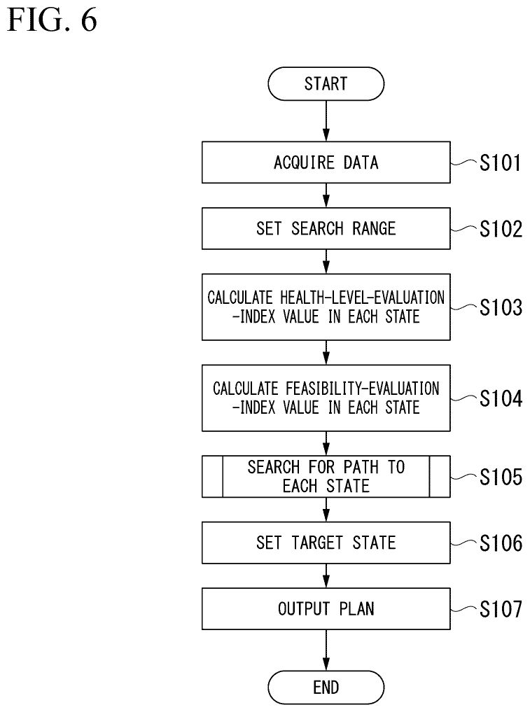

FIG. 6 is a diagram showing an example of a processing procedure for the planning device 100 to generate and output a plan.

In the processing of FIG. 6, the data acquisition unit 191 acquires various data for generating a plan (Step S101).

Next, the processing unit 190 sets a search range (Step S102).

Next, the health-level-evaluation-index-value acquisition unit 192 calculates the health-level-evaluation-index value for each state within the search range (Step S103).

Moreover, the feasibility-evaluation-index-value acquisition unit 193 calculates the feasibility-evaluation-index value for each state within the search range (Step S104).

Next, the path search unit 195 searches for paths from the initial state to each state within the search range using an evaluation function that uses the health-level-evaluation-index value for each state and the feasibility-evaluation-index value for each state (Step S105). The path search method used by the path search unit 195 is not limited to a specific one. For example, the path search unit 195 may perform path searching using the Dijkstra algorithm, but is not limited to this example.

Next, the target setting unit 194 sets a target state (Step S106). The target setting unit 194 may determine the state within the search range that is best evaluated as indicated by the health-level-evaluation-index value (that is, the state in which the health-level-evaluation-index value indicates the best health level) as the target state.

Next, the planning device 100 outputs a path from the initial state to the target state as a plan (Step S107). For example, the path search unit 195 may select a path to the target state from among the paths detected for each state within the search range, and treat it as a plan. The processing unit 190 then may control the display unit 120 to display the plan.

After Step S107, the planning device 100 ends the process of FIG. 6.

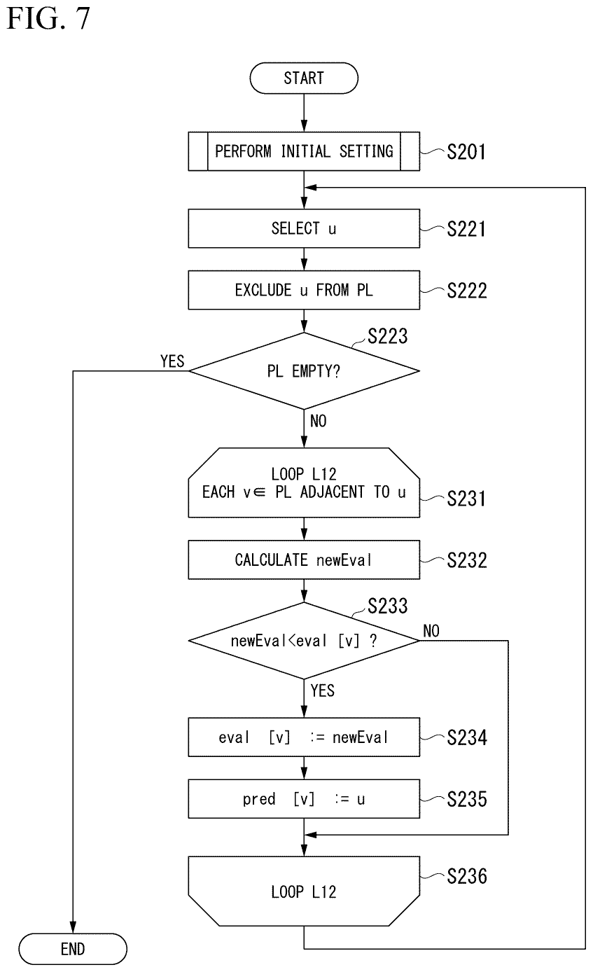

FIG. 7 is a diagram showing an example of a processing procedure for the path search unit 195 to perform path searching. FIG. 7 shows a case, as an example, where the path search unit 195 performs path searching using the Dijkstra algorithm. In the case where the path search unit 195 uses the Dijkstra algorithm, a cost that takes a positive value is used as the evaluation value for one state transition, as in the example of Expression (12).

The path search unit 195 performs the process of FIG. 7 in Step S105 of FIG. 6.

In the process of FIG. 7, the path search unit 195 performs initial setting for path searching (Step S201).

During the initial setting, the path search unit 195 sets the initial values for a list PL, the path evaluation index eval[v] for each state v, and the predecessor state pred[v] for each state v.

The path evaluation index eval[v] is a variable that indicates the evaluation value for the path from the initial state s to the state v.

The list PL is a list whose elements are states in which the value of the path evaluation index eval[v] is not yet determined.

The predecessor state pred[v] is a variable that indicates the state immediately before the state v (the state that directly transitions to state v) on the path from the initial state s to state v.

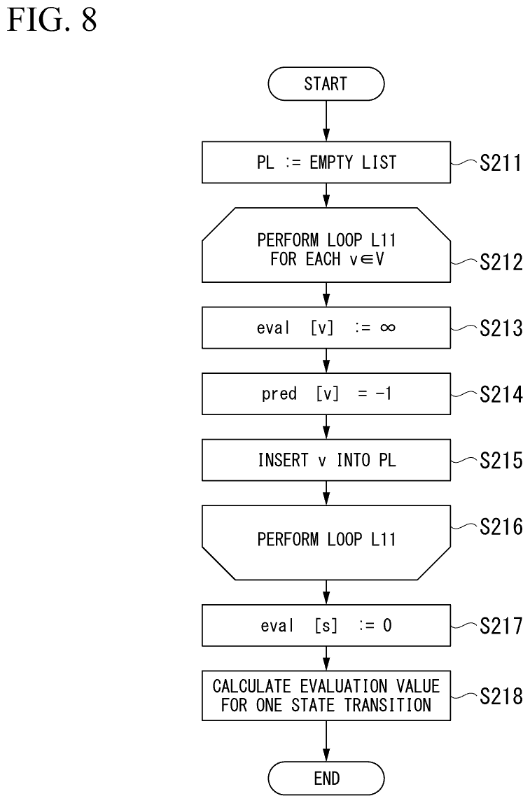

FIG. 8 is a diagram showing an example of a processing procedure for the path search unit 195 to perform initial setting for path searching. The path search unit 195 performs the process of FIG. 8 in Step S201 of FIG. 7.

In the process of FIG. 8, the path search unit 195 makes the list PL an empty list (Step S211).

Next, the path search unit 195 starts a loop L11 in which processing is performed for each state v included in the search range V (Step S212). A state that is a process target in the loop L11 is referred to as state v.

In the process in the loop L11, the path search unit 195 sets the value of the path evaluation index eval[v] to infinity (∞) (Step S213). The process in Step S203 can be expressed as eval[v]:=∞. The value set by the path search unit 195 to the path evaluation index eval[v] in Step S213 is not limited to infinity, but may be any value that is sufficiently large relative to the actually calculated value of the path evaluation index eval[v].

Moreover, the path search unit 195 sets the value of the predecessor state pred[v] to −1 (Step S214). The process in Step S204 can be expressed as pred[v]:=−1. The value of the predecessor state pred[v] being −1 indicates that the value of the predecessor state pred[v] is undetermined. If the predecessor state pred[v] is undetermined, this includes cases where state v has no predecessor state.

Furthermore, the path search unit 195 inserts the state v into the list PL (Step S215). In other words, the path search unit 195 includes the state v in the elements of the list PL.

Next, the path search unit 195 performs a termination process of the loop L11 (Step S216). Specifically, the path search unit 195 determines whether the process of the loop L11 has been performed for all states v included in the search range V. If it is determined that there is a state v for which the process of the loop L11 has not been performed, the path search unit 195 continues to perform the process of the loop L11 for the unprocessed state v. On the other hand, if it is determined that the process of the loop L11 has been performed for all states v included in the search range V, the path search unit 195 ends the loop L11.

If the loop L11 is ended in Step S216, the path search unit 195 sets the value of the path evaluation index eval[s] in the initial state s to 0 (Step S217). The process in Step S217 can be expressed as eval[s]:=0.

Through the processes in Step S213 and Step S217, the path search unit 195 initially sets the value of the path evaluation index eval[s] for the initial state s to 0, and sets the value of the path evaluation index eval[v] for the other states v to infinity.

Moreover, the path search unit 195 calculates an evaluation value for one state transition for each combination of two adjacent states within the search range (Step S218). In the case where two adjacent states are defined as a first state and a second state, and both a state transition from the first state to the second state and a state transition from the second state to the first state can occur, the path search unit 195 calculates an evaluation value for one state transition for each of these two state transitions.

The evaluation value for one state transition corresponds to the weight of an edge in the graph. In the Dijkstra algorithm, the path search unit 195 calculates the value of the path evaluation index eval[v] by accumulating the evaluation values for one state transition.

After Step S218, the path search unit 195 ends the process of FIG. 8. In such a case, the path search unit 195 ends the process of Step S201 in FIG. 7, and the processing proceeds to Step S221.

After Step S201 in FIG. 7, the path search unit 195 selects the state with the smallest value of the path evaluation index eval[v] from among the states v included in the list PL, and sets it as state u (Step S221). The process in Step S221 can be expressed as in Expression (19).

$$\begin{matrix}( Expression 19 ) & \\ u := {argmin}_{vϵPL} eval [ v ] & ( 19 )\end{matrix}$$

The state u can be considered as the state currently reached in the path searching. The state u is also referred to as the current state in the path searching.

Next, the path search unit 195 excludes the state u from the list PL (Step S222). In other words, the path search unit 195 deletes the state u from the elements of the list PL.

Next, the path search unit 195 determines whether or not the list PL is empty (Step S223). If the list PL is determined as not empty (Step S223: NO), the path search unit 195 starts a loop L12 in which processing is performed for each state v adjacent to the state u (Step S231). A state that is a process target in the loop L12 is referred to as state v.

In the process in loop L12, the path search unit 195 calculates a path evaluation index value when transitioning from state u to state v (Step S232). The path evaluation index value when transitioning from state u to state v is also represented as newEval.

The path search unit 195 calculates the path evaluation index value newEval for the transition from state u to state v by adding the evaluation value for one state transition from state u to state v to the value of the path evaluation index eval[u] in state u. If the evaluation value for one state transition from state u to state v is represented as xᵢ, the process in Step S232 can be expressed as in Expression (20).

$$\begin{matrix}( Expression 20 ) & \\ newEval := eval [ u ] + {x}_{i} & ( 20 )\end{matrix}$$

As described above, a cost that takes a positive value is used as the evaluation value xᵢ for one state transition.

Next, the path search unit 195 determines whether or not newEval<eval[v](Step S233).

If it is determined that newEval<eval[v] holds, the path search unit 195 updates the value of the path evaluation index eval[v] in state v to the path evaluation index value newEval when transitioning from state u to state v (Step S234). The process in Step S234 can be expressed as eval[v]:=newEval.

Furthermore, the path search unit 195 updates the predecessor state pred[v] of the state v to the state u (Step S235). The process in Step S235 can be expressed as pred[v]:=u.

Next, the path search unit 195 performs a termination process of the loop L12 (Step S236). Specifically, the path search unit 195 determines whether or not the process of the loop L12 has been performed for all states v adjacent to the current state u in the path searching. If it is determined that there is a state v for which the process of the loop L12 has not been performed, the path search unit 195 continues to perform the process of the loop L12 for the unprocessed state v. On the other hand, if it is determined that the process of the loop L12 has been performed for all states v adjacent to the current state u in the path searching, the path search unit 195 ends the loop L12.

If the path search unit 195 ends the loop L12 in Step S236, the processing returns to Step S221.

On the other hand, if the path search unit 195 determines in Step S233 that newEval≥eval[v] holds (Step S233: NO), the processing proceeds to Step S236.

Meanwhile, if it is determined in Step S223 that the list PL is empty (Step S223: YES), the path search unit 195 ends the processing of FIG. 7. In such a case, the path search unit 195 ends the process of Step S105 in FIG. 6, and the processing proceeds to Step S106.

After the target setting unit 194 sets the target state, the path search unit 195 may perform path searching. In such a case, the feasibility-evaluation-index-value acquisition unit 193 may acquire the feasibility-evaluation-index value when it becomes necessary in the path searching.

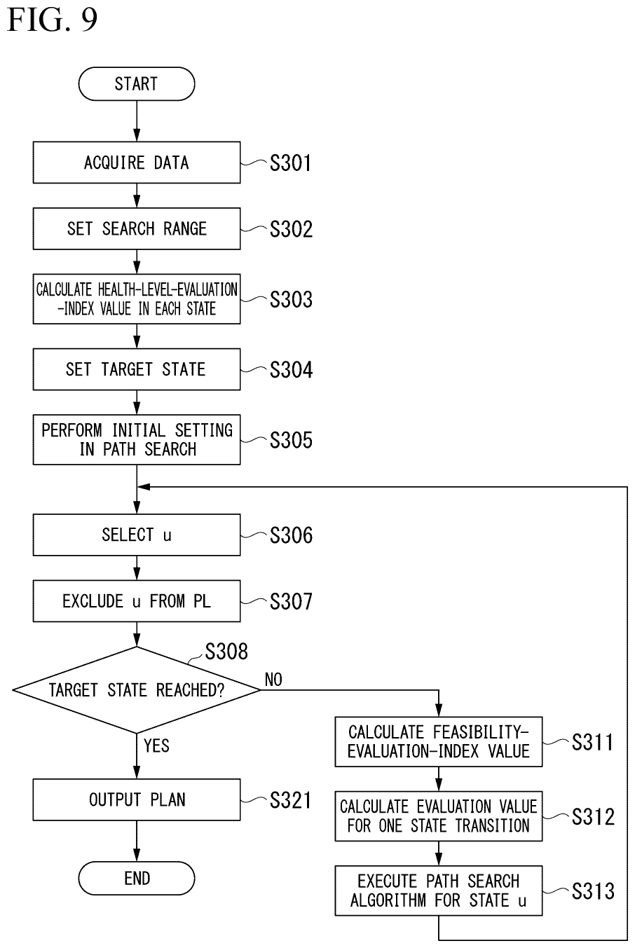

FIG. 9 is a diagram showing an example of a processing procedure by which the planning device 100 generates and outputs a plan when the target setting unit 194 sets a target state and then the path search unit 195 performs path searching.

Step S301 to Step S303 in FIG. 9 are similar to Step S101 to Step S103 in FIG. 6.

After Step S303, the target setting unit 194 sets a target state (Step S304). Step S304 is similar to Step S106 of FIG. 6.

The processing of Step S305 to Step S309 differs from the processing of Steps S104 to Step S106 in FIG. 6, FIG. 7, and FIG. 8 in that, during path searching, the feasibility-evaluation-index-value acquisition unit 193 calculates an evaluation value for a state transition, and the path search unit 195 calculates an evaluation value for one state transition, and in that the path search unit 195 terminates the path searching when the target state is reached. In other respects, the processing in Step S305 to Step S309 is similar to the processing of Step S104 to Step S106 in FIG. 6 and the processing in FIG. 7 and FIG. 8.

After Step S304, the path search unit 195 performs initial setting for path searching (Step S305). Step S305 is similar to Step S211 to Step S218 of FIG. 8.

Next, the path search unit 195 selects a state u from the states included in the list PL (Step S306). Step S306 is similar to Step S221 of FIG. 7. Next, the path search unit 195 excludes the state u from the list PL (Step S307). Step S307 is similar to Step S222 of FIG. 7.

Next, the path search unit 195 determines whether or not the target state has been reached (Step S308). Specifically, the path search unit 195 determines whether or not the state u matches the target state. If the path search unit 195 determines that the target state has not been reached (Step S308: NO), the feasibility-evaluation-index-value acquisition unit 193 acquires the feasibility-evaluation-index value for each state included in the list PL that is adjacent to state u (the current state in the path searching) (Step S311). If there is a state in which a feasibility-evaluation-index value has already been acquired, the acquired feasibility-evaluation-index value can be used, and the feasibility-evaluation-index-value acquisition unit 193 does not need to acquire the feasibility-evaluation-index value in that state again.

Next, the path search unit 195 calculates an evaluation value xᵢ for one state transition from the state u for each state adjacent to the state u (Step S312).

Next, the path search unit 195 executes a path search algorithm for the state u selected in Step S306 (Step S313). Step S313 is similar to Step S231 to Step S236 of FIG. 7. The feasibility-evaluation-index-value acquisition unit 193 and the path search unit 195 may perform the processes of Step S311 and Step S312 in a loop equivalent to the loop L12 in FIG. 7.

After Step S313, the process returns to Step S306.

Meanwhile, in Step S308, if the path search unit 195 determines that the target state has been reached (Step S308: YES), the planning device 100 outputs the plan (Step S321). Step S321 is similar to Step S107 of FIG. 6.

After Step S321, the planning device 100 ends the process of FIG. 9.

The planning device 100 may expand (broaden) the search range. For example, in the case where the subject determines the target value for the health-level-evaluation index, if there is no state that satisfies the target value within the search range, the planning device 100 may expand the search range.

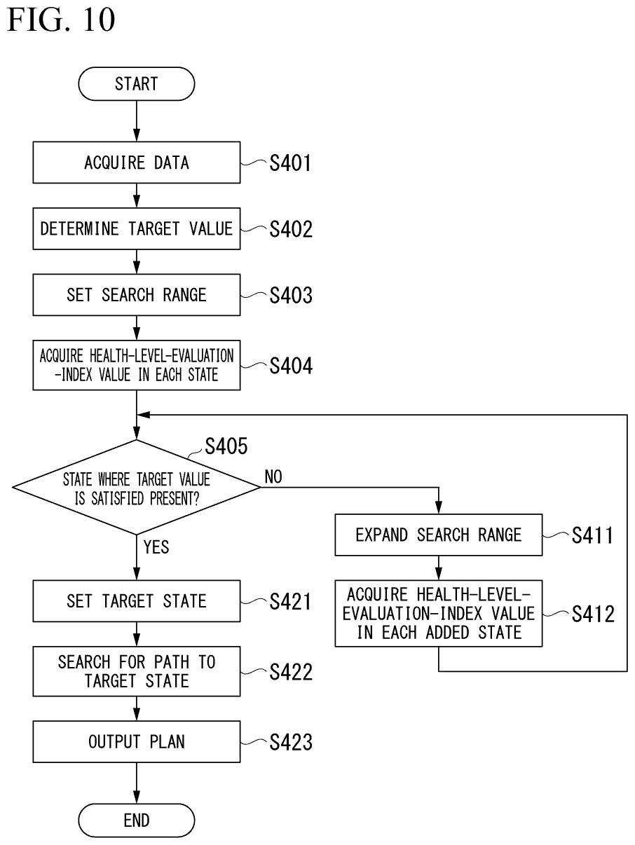

FIG. 10 is a diagram showing an example of a processing procedure for the planning device 100 to generate and output a plan, in the case of expanding the search range.

Step S401 of FIG. 10 is similar to Step S101 of FIG. 6.

After Step S401, the data acquisition unit 191 determines the target value for the health-level-evaluation index (Step S402). For example, the data acquisition unit 191 may set a target value received by the operation input unit 130 through a user operation as a target value in path searching.

Step S403 to Step S404 are similar to Step S102 to Step S103 in FIG. 6.

Next, the target setting unit 194 determines whether or not there is a state that satisfies the target value set in Step S402 among the states included in the search range (Step S405).

If the target setting unit 194 determines that there is no state that satisfies the target value (Step S405: NO), the processing unit 190 expands the search range (Step S411). For example, the processing unit 190 may broaden the range of each state identifying item value as the search range by a predetermined width. By expanding the search range, the number of states included in the search range increases.

Next, the health-level-evaluation-index-value acquisition unit 192 acquires the health-level-evaluation-index value for each state that has been newly included in the search range as a result of expanding the search range (Step S412).

After Step S412, the process returns to Step S405.

Meanwhile, if it is determined in Step S405 that there is a state within the search range that satisfies the target value set in Step S402 (Step S405: YES), the target setting unit 194 sets the state that is determined to satisfy the target value as the target state (Step S421). In the case where there are multiple states that satisfy the target value, the target setting unit 194 sets one of the multiple states as the target state.

Next, the path search unit 195 searches for a path from the initial state to the target state (Step S422). Step S422 is similar to Step S305 to Step S313 of FIG. 9.

As in the case of the processing in FIG. 6, the health-level-evaluation-index-value acquisition unit 192 and the path search unit 195 may calculate the health-level-evaluation-index value and the evaluation value for one state transition before executing the path search loop. For example, instead of the process corresponding to Step S311 in FIG. 9, the feasibility-evaluation-index-value acquisition unit 193 may acquire the feasibility-evaluation-index value for each state that has been newly included in the search range as a result of expanding the search range in Step S412. Moreover, instead of the process corresponding to Step S312 in FIG. 9, the path search unit 195 may calculate the evaluation value for one state transition for each state transition from and to each state that has been newly included in the search range as a result of expanding the search range in Step S412.

After Step S422, the planning device 100 outputs the plan (Step S423). The transition from Step S422 to Step S423 corresponds to the transition to Step S321 in the case of YES in Step S308 in FIG. 9. Step S423 is similar to Step S321 of FIG. 9.

After Step S423, the planning device 100 ends the process of FIG. 10.

The planning device 100 may generate a plan spanning multiple periods. The one period here is not limited to a specific one. For example, the planning device 100 may generate a plan spanning multiple years, with one year being one period, but the disclosure is not limited to this example.

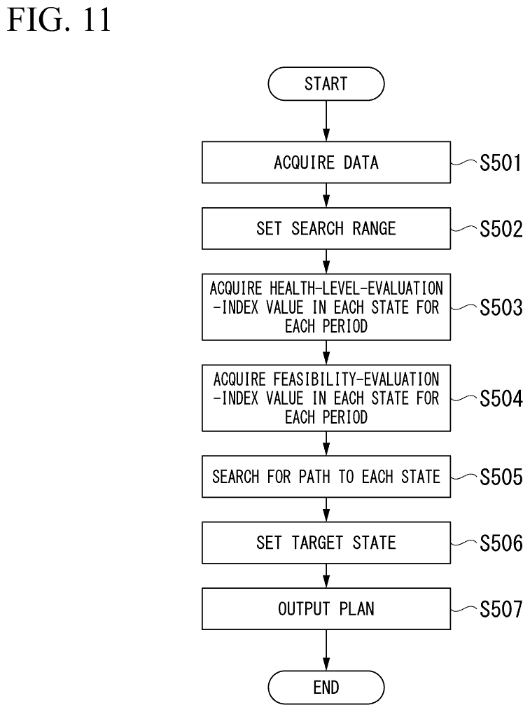

FIG. 11 is a diagram showing an example of a processing procedure for the planning device 100 to generate and output a plan that spans multiple periods.

In the processing of FIG. 11, the data acquisition unit 191 acquires various data for generating a plan (Step S501). In particular, the data acquisition unit 191 acquires data for multiple periods. For instance, as the subject grows older, the data to be referenced for plan generation may vary across different periods. When the data acquisition unit 191 acquires data spanning multiple periods, the planning device 100 is expected to generate a plan with improved accuracy.

Step S501 is similar to Step S101 in FIG. 6, except that the data acquisition unit 191 acquires data for multiple periods.

Next, the processing unit 190 sets a search range (Step S502). Step S502 is similar to Step S102 of FIG. 6.

Next, the health-level-evaluation-index-value acquisition unit 192 acquires, for each period, the health-level-evaluation-index value for each state within the search range (Step S503).

By providing a state for each period, the transition between periods can be represented as a state transition, and processing can be performed in the same manner as in the case where the planning target period is regarded as a single period, as in the example of FIG. 6. It should be noted that, with respect to state transitions due to the transitions between periods, a constraint is imposed such that transitions cannot occur from a state belonging to a new period to a state belonging to an old period.

Furthermore, the health-level-evaluation-index-value acquisition unit 192 acquires the health-level-evaluation-index value for each state within the search range for each period, and it is thereby expected that the planning device 100 will be able to generate a plan with improved accuracy.

Next, the feasibility-evaluation-index-value acquisition unit 193 acquires the feasibility-evaluation-index value in each state within the search range for each period (Step S504). The feasibility-evaluation-index-value acquisition unit 193 acquires the feasibility-evaluation-index value for each state within the search range for each period, and it is thereby expected that the planning device 100 will be able to generate a plan with improved accuracy.

Step S505 to Step S507 are similar to Step S105 to Step S107 in FIG. 6. It should be noted that, in the case where the plan is to necessarily span a predetermined period, the target setting unit 194 is configured to select a target state from the last period among the multiple periods that are the subject of the plan.

After Step S507, the planning device 100 ends the process of FIG. 11.

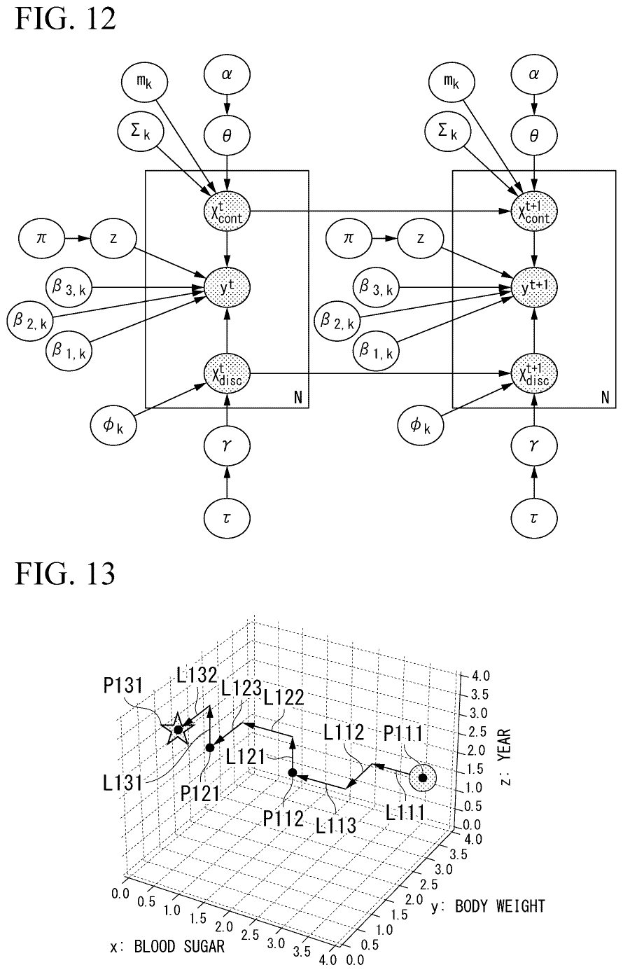

FIG. 12 is a diagram showing an example of a feasibility-evaluation-index model used by the feasibility-evaluation-index-value acquisition unit 193 when the planning device 100 generates a plan that spans multiple periods.

FIG. 12 shows a case, as an example, in which the planning device 100 generates a plan spanning two periods, where t indicates the older of the two periods and t+1 indicates the newer of the two periods.

In the example of FIG. 12, the model in the example of FIG. 5 is provided for each period. The parameter X_{cont}^{t} that takes a continuous value in period t and the parameter X_{cont}^{t+1} that takes a continuous value in period t+1 are assumed to be related, not independent. Furthermore, the parameter X_{disc}^{t} that takes a discrete value in period t and the parameter X_{disc}^{t+1} that takes a discrete value in period t+1 are assumed to be related, not independent.

The relationship between the parameter X_{cont}^{t} and the parameter X_{cont}^{t+1} can be expressed as in Expression (21).

$$\begin{matrix}( Expression 21 ) & \\ p ( {X}_{cont}^{t + 1} \vert {X}_{cont}^{t} ) = N ( {X}_{cont}^{t + 1} \vert {AX}_{cont}^{t} , σ I ) & ( 21 )\end{matrix}$$

Expression (21) shows that the conditional distribution of the parameter X_{cont}^{t+1} that takes a continuous value in period t+1 follows a Gaussian distribution, given the parameter X_{cont}^{t} that takes a continuous value from period t. Here, AX_{cont}^{t} represents the expected value of the Gaussian distribution, and σI represents the variance-covariance matrix of the Gaussian distribution. Here, A represents an M×1 dimensional matrix, and σI represents an M dimensional identity matrix.

The relationship between the parameters X_{disc}^{t} and X_{disc}^{t+1} can be expressed by the transition probability shown in Expression (22).

$$\begin{matrix}( Expression 22 ) & \\ p ( {X}_{disc}^{t + 1} \vert {X}_{disc}^{t} ) & ( 22 )\end{matrix}$$

FIG. 13 is a diagram showing an example of displaying a plan that spans multiple periods on the display unit 120. FIG. 13 shows a case, as an example, in which the state identifying items are weight and blood sugar, and the period is a year. The x-axis of the graph in FIG. 13 represents blood sugar, the y-axis represents body weight, and the z-axis represents year. In the example of FIG. 13, both body weight and blood sugar are shown as differences from the reference values.

Point P111 indicates the initial state.

Lines L111, L112 and L113 all indicate the paths in the first year.

Point P112 indicates the target state in the first year. Point P112 can be considered as an intermediate target state in the plan.

Line L121 indicates a state transition corresponding to the transition from the first year to the second year.

Lines L122 and L123 both indicate the paths in the second year.

Point P121 indicates the target state in the second year. Point P121 can be considered as an intermediate target state in the plan.

Line L131 indicates a state transition corresponding to the transition from the second year to the third year.

Line L132 indicates the path in the third year.

Point P131 indicates the initial state.

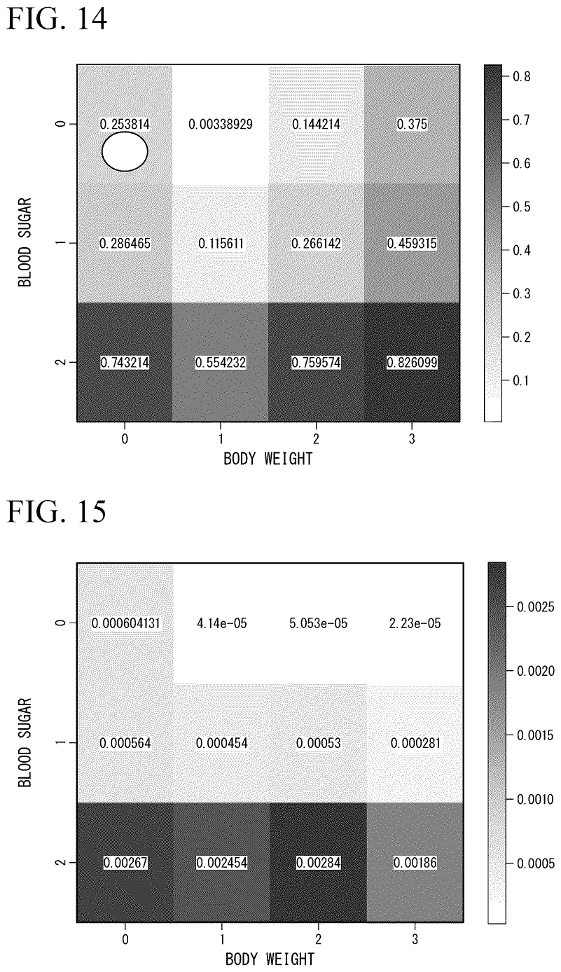

FIG. 14 is a diagram showing a first example of displaying data for each period on the display unit 120. FIG. 14 shows data for the first (older) of the two periods covered by the plan.

FIG. 14 shows a case, as an example, where the state identifying items are weight and blood sugar, with the horizontal direction (horizontal direction of the figure) corresponding to the weight values and the vertical direction (vertical direction of the figure) corresponding to the blood sugar values.

Moreover, along the horizontal direction, column numbers are shown as 0, 1, 2, and 3, increasing from left to right. A smaller column number (and therefore a position further to the left in FIG. 14) indicates a higher body weight. Along the vertical direction, row numbers are shown as 0, 1, and 2, increasing from top to bottom. A smaller row number (and therefore a position higher up in FIG. 14) indicates a higher blood sugar value.

The display unit 120 may show representative values or sections for body weight in each column, and representative values or sections for blood sugar in each row.

Also, in the example of FIG. 14, as an evaluation index value for each state, an occurrence probability+1-diabetes onset risk (a sum of an occurrence probability of a combination of a body weight value and a blood sugar value assigned to each state, and a value obtained by subtracting the diabetes onset risk from 1) is shown.

Also, in the example of FIG. 14, a state at row 0 and column 0 (the leftmost and uppermost state in FIG. 14) is an initial state.

FIG. 15 is a diagram showing a second example of displaying data for each period on the display unit 120. FIG. 15 shows data for the second (newer) of the two periods covered by the plan.

FIG. 15 shows a case, as an example, where the state identifying items are weight and blood sugar, with the horizontal direction (horizontal direction of the figure) corresponding to the body weight values and the vertical direction (vertical direction of the figure) corresponding to the blood sugar values.

Moreover, along the horizontal direction, column numbers are shown as 0, 1, 2, and 3, increasing from left to right. A smaller column number (and therefore a position further to the left in FIG. 15) indicates a higher body weight. Along the vertical direction, row numbers are shown as 0, 1, and 2, increasing from top to bottom. A smaller row number (and therefore a position higher up in FIG. 15) indicates a higher blood sugar value.

The display unit 120 may show representative values or sections for body weight in each column, and representative values or sections for blood sugar in each row.

Also, in the example of FIG. 15, as an evaluation index value for each state, an occurrence probability+1-diabetes onset risk (a sum of an occurrence probability of a combination of a body weight value and a blood sugar value assigned to each state, and a value obtained by subtracting the diabetes onset risk from 1) is shown.

FIG. 14 and FIG. 15 show data for the same range of combinations of body weight and blood sugar values in the first and second periods, respectively. Thus, the planning device 100 is expected to be able to generate a plan with comparatively high accuracy by acquiring data for each period regarding states corresponding to the same state identifying item value.

As described above, the feasibility evaluation index value acquisition unit 193 acquires, for each state identified by values of one or more items correlated with a health-level-evaluation-index value, a feasibility evaluation index value quantitatively indicating feasibility of each individual state.

The health-level-evaluation-index-value acquisition unit 192 acquires a health-level-evaluation-index value for each individual state.

The path search unit 195 searches for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

According to the planning device 100, it is expected to enable a subject who executes a plan for gradually changing combinations of quantitative values that are considered to be state identifying items, to confirm the result of executing the plan.

In particular, according to the planning device 100, by using an evaluation value for each individual state transition that indicates a better evaluation as the health-level-evaluation-index value in the transition-destination state shows a better evaluation than the health-level-evaluation-index value in the transition-source state, it is expected that the subject can confirm changes in the health-level-evaluation-index value at the time of state transitions.

In addition, according to the planning device 100, by using an evaluation value that indicates a better evaluation as the transition-destination state shows better feasibility, it is expected that the subject can comparatively easily execute a plan.

Moreover, the path search unit 195 uses the health-level-evaluation-index value in the transition-destination state as an evaluation value that indicates a better evaluation where the health-level-evaluation-index value in the transition-destination state indicates a better evaluation relative to the health-level-evaluation-index value in the transition-source state.

According to the planning device 100, it is expected that a state transition for which an evaluation indicated by a health-level-evaluation-index value becomes better can be selected with a comparatively simple calculation.

Moreover, the target setting unit 194 determines a target state in the plan based on the health-level-evaluation-index value for each state.

The path search unit 195 searches for a path from an initial state in the plan to the target state determined by the target setting unit 194.

According to the planning device 100, it is possible to end path searching when the target state is reached. In this regard, according to the planning device 100, it is expected to require a comparatively small amount of calculation.

Moreover, the path search unit 195 searches for a path from an initial state in the plan to each individual state other than the initial state.

Among the paths detected by the path search unit 195, a path from the initial state to a state set as a target state is used as a path from the initial state to the target state in the plan. According to the planning device 100, path searching can be performed before a target state is set. In this respect, according to the planning device 100, it is expected to shorten the time from setting the target state to generating the plan.

Moreover, the health-level-evaluation-index-value acquisition unit 192 acquires the health-level-evaluation-index value for each state that can be reached within a predetermined number of state transitions from an initial state in the plan.

According to the planning device 100, the search range can be specified by specifying the number of state transitions from the initial state. In this respect, according to the planning device 100, the search range can be specified comparatively easily.

Furthermore, the path search unit 195 searches for a path from an initial state to a target state in a plan using an evaluation value for a path involving one or more state transitions, the evaluation value being calculated using evaluation values for individual state transitions, each of which is determined as a weighted sum of an evaluation value indicating, as an evaluation for a single state transition from a transition-source state to a transition-destination state, a better evaluation where the transition-destination state has higher feasibility, and an evaluation value indicating a better evaluation where the health-level-evaluation-index value in the transition-destination state shows a better evaluation relative to the health-level-evaluation-index value in the transition-source state.

According to the planning device 100, with a relatively simple process of adjusting weights, it is possible to adjust a balance between the ease of plan execution with the likelihood of the subject being able to see results from plan execution.

Moreover, the health-level-evaluation-index-value acquisition unit 192 calculates the health-level-evaluation-index value using a model in which, among parameters of the health-level-evaluation-index-value, a parameter that takes values following a continuous distribution and a parameter that take values following a discrete distribution have mutually different latent variables as parameters.

According to the planning device 100, it is expected that the feasibility evaluation index value can be calculated with comparatively high accuracy.

Moreover, the state in a plan is identified using values of one or more items correlated with a health-level-evaluation-index value and any one of a plurality of periods.

The feasibility-evaluation-index-value acquisition unit 193 acquires the feasibility-evaluation-index value for each value of one or more items correlated with the health-level-evaluation-index value and for each period.

The health-level-evaluation-index-value acquisition unit 192 acquires the health-level-evaluation-index value for each value of one or more items correlated with the health-level-evaluation-index-value and for each period.

According to the planning device 100, it is expected that a plan can be generated with comparatively high accuracy in that the health-level-evaluation-index value and the feasibility-evaluation-index value are acquired for each period.

Moreover, the health-level-evaluation-index-value acquisition unit 192 calculates the health-level-evaluation-index value for each individual state by using a trained model that outputs, upon receiving input of values of one or more items identifying the state, the health-level-evaluation-index value for the defined state.

According to the planning device 100, a model that outputs a health-level-evaluation-index values is acquired through learning, and therefore it is possible to acquire a health-level-evaluation-index value that reflects statistical data. In this respect, according to the planning device 100, it is expected to be possible to generate a plan with comparatively high accuracy.

Moreover, one or more items used to identify the state are selected from the measurement target items for a subject of the plan, based on correlation between each measurement target item and the health-level-evaluation-index value.

According to the planning device 100, the items used to identify the state (state identifying items) are selected based on the correlation between the measurement target items and the health-level-evaluation-index values, and therefore it is expected that changes in the state identifying item values will influence the health-level-evaluation-index values. In this respect, according to the planning device 100, it is expected to be possible to generate a plan with comparatively high accuracy.

### Second Example Embodiment

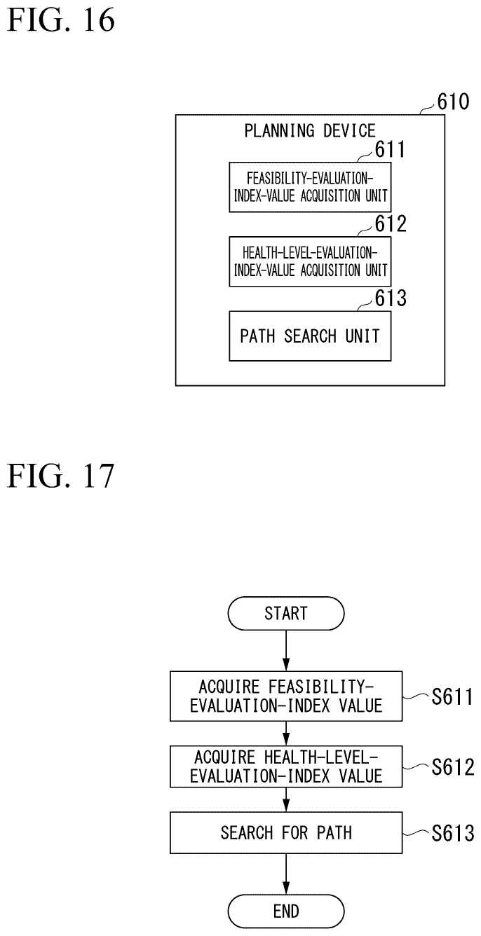

FIG. 16 is a diagram showing a configuration example of a planning device according to at least one of the example embodiments. In the configuration shown in FIG. 16, a planning device 610 includes a feasibility-evaluation-index-value acquisition unit 611, a health-level-evaluation-index-value acquisition unit 612, and a path search unit 613.

With such a configuration, the feasibility-evaluation-index-value acquisition unit 611 acquires, for each state identified by values of one or more items correlated with a health-level-evaluation-index value, a feasibility evaluation index value quantitatively indicating feasibility of each individual state.

The health-level-evaluation-index-value acquisition unit 612 acquires the health-level-evaluation-index value for each individual state.

The path search unit 613 searches for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

The feasibility-evaluation-index-value acquisition unit 611 corresponds to an example of the feasibility-evaluation-index-value acquisition means. The health-level-evaluation-index-value acquisition unit 612 is an example of the health-level-evaluation-index-value acquisition means. The path search unit 613 corresponds to an example of the path search means.

According to the planning device 610, it is expected to enable a subject who executes a plan for gradually changing combinations of quantitative values that are considered to be state identifying items (items used to identify states), to confirm the result of executing the plan.

In particular, according to the planning device 610, by using an evaluation value for each individual state transition that indicates a better evaluation as the health-level-evaluation-index value in the transition-destination state shows a better evaluation than the health-level-evaluation-index value in the transition-source state, it is expected that the subject can confirm changes in the health-level-evaluation-index value at the time of state transitions.

In addition, according to the planning device 610, by using an evaluation value that indicates a better evaluation as the transition-destination state shows better feasibility, it is expected that the subject can comparatively easily execute a plan.

### Third Example Embodiment

FIG. 17 is a diagram showing an example of a processing procedure in a planning method according to at least one of the example embodiments. The planning method shown in FIG. 17 includes: acquiring a feasibility-evaluation-index value (Step S611); acquiring a health-level-evaluation-index value (Step S612); and searching for a path (Step S613).

In the step of acquiring a feasibility-evaluation-index value (Step S611), a computer acquires, for each state identified by values of one or more items correlated with a health-level-evaluation-index value, a feasibility evaluation index value quantitatively indicating feasibility of each individual state.

In the step of acquiring a health-level-evaluation-index value (Step S612), the computer acquires a health-level-evaluation-index value for each individual state.

In the step of searching for a path (Step S613), the computer searches for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

According to the planning method shown in FIG. 17, it is expected to enable a subject who executes a plan for gradually changing combinations of quantitative values that are considered to be state identifying items (items used to identify states), to confirm the result of executing the plan.

In particular, according to the planning method shown in FIG. 17, by using an evaluation value for each individual state transition that indicates a better evaluation as the health-level-evaluation-index value in the transition-destination state shows a better evaluation than the health-level-evaluation-index value in the transition-source state, it is expected that the subject can confirm changes in the health-level-evaluation-index value at the time of state transitions.

Also, according to the planning method shown in FIG. 17, by using an evaluation value that indicates a better evaluation as the transition-destination state shows better feasibility, it is expected that the subject can comparatively easily execute a plan.

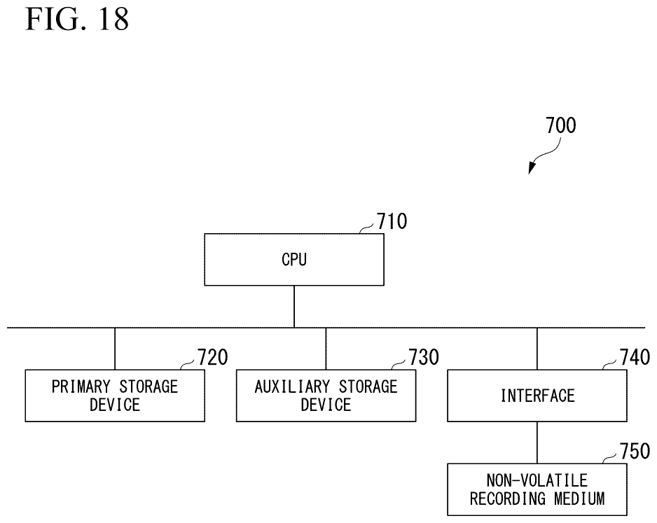

FIG. 18 is a diagram showing a configuration example of a computer according to at least one of the example embodiments. In the configuration shown in FIG. 18, a computer 700 includes a CPU 710, a primary storage device 720, an auxiliary storage device 730, an interface 740, and a non-volatile recording medium 750.

One or more of the planning device 100 and the planning device 610 or part thereof may be implemented in the computer 700. In such a case, operations of the respective processing units described above are stored in the auxiliary storage device 730 in the form of a program. The CPU 710 reads out the program from the auxiliary storage device 730, loads it on the primary storage device 720, and executes the processing described above according to the program. Moreover, the CPU 710 secures, according to the program, memory storage regions corresponding to the respective storage units mentioned above, in the primary storage device 720. Communication between each device and other devices is executed by the interface 740 having a communication function and communicating under the control of the CPU 710. The interface 740 also has a port for the non-volatile recording medium 750, and reads information from the non-volatile recording medium 750 and writes information to the non-volatile recording medium 750.

In the case where the planning device 100 is implemented in the computer 700, operations of the processing unit 190 and each component thereof are stored in the form of a program in the auxiliary storage device 730. The CPU 710 reads out the programs from the auxiliary storage device 730, loads them onto the primary storage device 720, and executes the processes described above, according to the programs.

Also, the CPU 710 secures a memory storage region in the primary storage device 720 for the storage unit 180, according to the program. Communication with another device performed by the communication unit 110 is executed by the interface 740 having a communication function and operating under the control of the CPU 710. Display of images performed by the display unit 120 is executed by the interface 740 having a display device and displaying various images under the control of the CPU 710. User operations are accepted through the operation input unit 130 by the interface 740 having an input device and accepting user operations under control of the CPU 710.

In the case where the planning device 610 is implemented in the computer 700, operations of the feasibility-evaluation-index-value acquisition unit 611, the health-level-evaluation-index-value acquisition unit 612, and the path search unit 613 are stored in the form of programs in the auxiliary storage device 730. The CPU 710 reads out the programs from the auxiliary storage device 730, loads them onto the primary storage device 720, and executes the processes described above, according to the programs.

Moreover, the CPU 710 secures a memory storage region in the primary storage device 720 for the processing to be performed by the planning device 610, according to the program. Communication with other devices performed by the planning device 610 is executed by the interface 740 having a communication function and operating under the control of the CPU 710. Interaction between the planning device 610 and the user is executed by the interface 740 having an input device and an output device, presenting information to the user through the output device under the control of the CPU 710, and accepting user operations through the input device.

Any one or more of the programs described above may be recorded in the non-volatile recording medium 750. In such a case, the interface 740 may read the program from the non-volatile recording medium 750. Then, the CPU 710 directly executes the program read by the interface 740, or it may be temporarily stored in the primary storage device 720 or the auxiliary storage device 730 and then executed.

It should be noted that a program for executing some or all of the processes performed by the planning device 100 and the planning device 610 may be recorded on a computer-readable recording medium, and the program recorded on the recording medium may be read into and executed on a computer system, to thereby perform the processing of each unit. The “computer system” here includes an OS (operating system) and hardware such as peripheral devices.

Moreover, the “computer-readable recording medium” referred to here refers to a portable medium such as a flexible disk, a magnetic optical disk, a ROM (Read Only Memory), and a CD-ROM (Compact Disc Read Only Memory), or a storage device such as a hard disk built into a computer system. The above program may be a program for realizing a part of the functions described above, and may be a program capable of realizing the functions described above in combination with a program already recorded in a computer system.

While the present disclosure has been described above with reference to the example embodiments, the present disclosure is not limited to the example embodiments described above. Various modifications that can be understood by those skilled in the art may be made to the configurations and/or details of the present disclosure, without departing from the scope of the disclosure. Furthermore, the example embodiments described above may be combined with another example embodiment as appropriate.

The whole or part of the example embodiments disclosed above can be described as, but not limited to, the following supplementary notes.

### (Supplementary Note 1)

A planning device comprising:
a memory configured to store instructions; and
a processor configured to execute the instructions to:
acquire, for each state identified by a value of one or more items correlated with a health-level-evaluation-index value, a feasibility evaluation index value quantitatively indicating feasibility of the state;
acquire a health-level-evaluation-index value for each state; and
search for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

### (Supplementary Note 2)

The planning device according to supplementary note 1, wherein the processor is configured to execute the instructions to use the health-level-evaluation-index value in the transition-destination state as an evaluation value that indicates a better evaluation where the health-level-evaluation-index value in the transition-destination state indicates a better evaluation relative to the health-level-evaluation-index value in the transition-source state.

### (Supplementary Note 3)

The planning device according to supplementary notes 1 or 2,
wherein the processor is configured to execute the instructions to determine a target state in the plan based on the health-level-evaluation-index value for each state, and
wherein the processor is configured to execute the instructions to search for a path from an initial state in the plan to the determined target state.

### (Supplementary Note 4)

The planning device according to supplementary notes 1 or 2,
wherein the processor is configured to execute the instructions to search for a path from the initial state in the plan to each state other than the initial state, and
among the detected paths, a path from the initial state to a state set as a target state is used as a path from the initial state to the target state in the plan.

### (Supplementary Note 5)

The planning device according to any one of supplementary notes 1 to 4, wherein the processor is configured to execute the instructions to acquire the health-level-evaluation-index value for each state that are reachable within a predetermined number of state transitions from the initial state in the plan.

### (Supplementary Note 6)

The planning device according to any one of supplementary notes 1 to 5, wherein the processor is configured to execute the instructions to search for the path from the initial state to the target state in the plan using an evaluation value for a path involving one or more state transitions, the evaluation value being calculated using evaluation values for individual state transitions, each of which is determined as a weighted sum of an evaluation value indicating, as an evaluation for a single state transition from a transition-source state to a transition-destination state, a better evaluation where the transition-destination state has higher feasibility, and an evaluation value indicating a better evaluation where the health-level-evaluation-index value in the transition-destination state shows a better evaluation relative to the health-level-evaluation-index value in the transition-source state.

### (Supplementary Note 7)

The planning device according to any one of supplementary notes 1 to 6, wherein the processor is configured to execute the instructions to calculate the health-level-evaluation-index value using a model that has, as parameters, a parameter that takes values following a continuous distribution and a parameter that take values following a discrete distribution have mutually different latent variables, among parameters of the health-level-evaluation-index-value.

### (Supplementary Note 8)

The planning device according to any one of supplementary notes 1 to 7,
wherein the state is identified using a value of one or more items correlated with a health-level-evaluation-index value and any one of a plurality of periods,
wherein the processor is configured to execute the instructions to acquire the feasibility-evaluation-index value for each value of the one or more items correlated with the health-level-evaluation-index value and for each period, and
wherein the processor is configured to execute the instructions to acquire the health-level-evaluation-index value for each value of the one or more items correlated with the health-level-evaluation-index-value and for each period.

### (Supplementary Note 9)

The planning device according to any one of supplementary notes 1 to 8, wherein the processor is configured to execute the instructions to calculate the health-level-evaluation-index value for each state by using a trained model that outputs, upon receiving an input of a value of one or more items identifying the state, the health-level-evaluation-index value for the defined state.

### (Supplementary Note 10)

The planning device according to any one of supplementary notes 1 to 9, wherein the processor is configured to execute the instructions to select one or more items used to identify the state from measurement target items for a subject of the plan, based on correlation between each measurement target item and the health-level-evaluation-index value.

### (Supplementary Note 11)

A planning method executed by a computer, the method comprising:
acquiring, for each state identified by a value of one or more items correlated with a health-level-evaluation-index value, a feasibility evaluation index value quantitatively indicating feasibility of the state;
acquiring a health-level-evaluation-index value for each state; and
searching for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

### (Supplementary Note 12)

The planning method according to supplementary note 11, wherein searching for the path comprises using the health-level-evaluation-index value in the transition-destination state as an evaluation value that indicates a better evaluation where the health-level-evaluation-index value in the transition-destination state indicates a better evaluation relative to the health-level-evaluation-index value in the transition-source state.

### (Supplementary Note 13)

The planning method according to supplementary notes 11 or 12, further comprising
determining a target state in the plan based on the health-level-evaluation-index value for each state, and
wherein searching for the path comprises searching for a path from an initial state in the plan to the determined target state.

### (Supplementary Note 14)

The planning method according to supplementary notes 11 or 12,
wherein searching for the path comprises searching for a path from the initial state in the plan to each state other than the initial state, and
wherein, among the detected paths, a path from the initial state to a state set as a target state is used as a path from the initial state to the target state in the plan.

### (Supplementary Note 15)

The planning method according to any one of supplementary notes 11 to 14, wherein acquiring the health-level-evaluation-index value comprises acquiring the health-level-evaluation-index value for each state that are reachable within a predetermined number of state transitions from the initial state in the plan.

### (Supplementary Note 16)

The planning method according to any one of supplementary notes 11 to 15, wherein searching for the path comprises searching for the path from the initial state to the target state in the plan using an evaluation value for a path involving one or more state transitions, the evaluation value being calculated using evaluation values for individual state transitions, each of which is determined as a weighted sum of an evaluation value indicating, as an evaluation for a single state transition from a transition-source state to a transition-destination state, a better evaluation where the transition-destination state has higher feasibility, and an evaluation value indicating a better evaluation where the health-level-evaluation-index value in the transition-destination state shows a better evaluation relative to the health-level-evaluation-index value in the transition-source state.

### (Supplementary Note 17)

The planning method according to any one of supplementary notes 11 to 16, wherein acquiring the health-level-evaluation-index value comprises calculating the health-level-evaluation-index value using a model that has, as parameters, a parameter that takes values following a continuous distribution and a parameter that take values following a discrete distribution have mutually different latent variables, among parameters of the health-level-evaluation-index-value.

### (Supplementary Note 18)

The planning method according to any one of supplementary notes 11 to 17,
wherein the state is identified using a value of one or more items correlated with a health-level-evaluation-index value and any one of a plurality of periods,
wherein acquiring the feasibility-evaluation-index value comprises acquiring the feasibility-evaluation-index value for each value of the one or more items correlated with the health-level-evaluation-index value and for each period, and
wherein acquiring the health-level-evaluation-index value comprises acquiring the health-level-evaluation-index value for each value of the one or more items correlated with the health-level-evaluation-index-value and for each period.

### (Supplementary Note 19)

The planning method according to any one of supplementary notes 11 to 18, wherein acquiring the health-level-evaluation-index value comprises calculating the health-level-evaluation-index value for each state by using a trained model that outputs, upon receiving an input of a value of one or more items identifying the state, the health-level-evaluation-index value for the defined state.

### (Supplementary Note 20)

The planning method according to any one of supplementary notes 11 to 19, further comprising selecting one or more items used to identify the state from measurement target items for a subject of the plan, based on correlation between each measurement target item and the health-level-evaluation-index value.

### (Supplementary Note 21)

A non-transitory computer-readable recording medium that stores a program for causing a computer to execute:
acquiring, for each state identified by a value of one or more items correlated with a health-level-evaluation-index value, a feasibility evaluation index value quantitatively indicating feasibility of the state;
acquiring a health-level-evaluation-index value for each state; and
searching for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

### (Supplementary Note 22)

The recording medium according to supplementary note 21, wherein searching for the path comprises using the health-level-evaluation-index value in the transition-destination state as an evaluation value that indicates a better evaluation where the health-level-evaluation-index value in the transition-destination state indicates a better evaluation relative to the health-level-evaluation-index value in the transition-source state.

### (Supplementary Note 23)

The recording medium according to supplementary notes 21 or 22,
wherein the program further causes the computer to execute determining a target state in the plan based on the health-level-evaluation-index value for each state, and
wherein searching for the path comprises searching for a path from an initial state in the plan to the determined target state.

### (Supplementary Note 24)

The recording medium according to supplementary notes 21 or 22,
wherein searching for the path comprises searching for a path from the initial state in the plan to each state other than the initial state, and
wherein, among the detected paths, a path from the initial state to a state set as a target state is used as a path from the initial state to the target state in the plan.

### (Supplementary Note 25)

The recording medium according to any one of supplementary notes 21 to 24, wherein acquiring the health-level-evaluation-index value comprises acquiring the health-level-evaluation-index value for each state that are reachable within a predetermined number of state transitions from the initial state in the plan.

### (Supplementary Note 26)

The recording medium according to any one of supplementary notes 21 to 25, wherein searching for the path comprises searching for the path from the initial state to the target state in the plan using an evaluation value for a path involving one or more state transitions, the evaluation value being calculated using evaluation values for individual state transitions, each of which is determined as a weighted sum of an evaluation value indicating, as an evaluation for a single state transition from a transition-source state to a transition-destination state, a better evaluation where the transition-destination state has higher feasibility, and an evaluation value indicating a better evaluation where the health-level-evaluation-index value in the transition-destination state shows a better evaluation relative to the health-level-evaluation-index value in the transition-source state.

### (Supplementary Note 27)

The recording medium according to any one of supplementary notes 21 to 26, wherein acquiring the health-level-evaluation-index value comprises calculating the health-level-evaluation-index value using a model that has, as parameters, a parameter that takes values following a continuous distribution and a parameter that take values following a discrete distribution have mutually different latent variables, among parameters of the health-level-evaluation-index-value.

### (Supplementary Note 28)

The recording medium according to any one of supplementary notes 21 to 27,
wherein the state is identified using a value of one or more items correlated with a health-level-evaluation-index value and any one of a plurality of periods,
wherein acquiring the feasibility-evaluation-index value comprises acquiring the feasibility-evaluation-index value for each value of the one or more items correlated with the health-level-evaluation-index value and for each period, and
wherein acquiring the health-level-evaluation-index value comprises acquiring the health-level-evaluation-index value for each value of the one or more items correlated with the health-level-evaluation-index-value and for each period.

### (Supplementary Note 29)

The recording medium according to any one of supplementary notes 21 to 28, wherein acquiring the health-level-evaluation-index value comprises calculating the health-level-evaluation-index value for each state by using a trained model that outputs, upon receiving an input of a value of one or more items identifying the state, the health-level-evaluation-index value for the defined state.

### (Supplementary Note 30)

The recording medium according to any one of supplementary notes 21 to 29, wherein the program further causes the computer to execute selecting one or more items used to identify the state from measurement target items for a subject of the plan, based on correlation between each measurement target item and the health-level-evaluation-index value.

What is claimed is:

1. A planning device comprising:
a memory configured to store instructions; and
a processor configured to execute the instructions to:
acquire, for each state identified by a value of one or more items correlated with a health-level-evaluation-index value, a feasibility evaluation index value quantitatively indicating feasibility of the state;
acquire a health-level-evaluation-index value for each state; and
search for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

2. The planning device according to claim 1, wherein the processor is configured to execute the instructions to use the health-level-evaluation-index value in the transition-destination state as an evaluation value that indicates a better evaluation where the health-level-evaluation-index value in the transition-destination state indicates a better evaluation relative to the health-level-evaluation-index value in the transition-source state.

3. The planning device according to claim 1,
wherein the processor is configured to execute the instructions to determine a target state in the plan based on the health-level-evaluation-index value for each state, and
wherein the processor is configured to execute the instructions to search for a path from an initial state in the plan to the determined target state.

4. The planning device according to claim 1,
wherein the processor is configured to execute the instructions to search for a path from the initial state in the plan to each state other than the initial state, and
among the detected paths, a path from the initial state to a state set as a target state is used as a path from the initial state to the target state in the plan.

5. The planning device according to claim 1, wherein the processor is configured to execute the instructions to acquire the health-level-evaluation-index value for each state that are reachable within a predetermined number of state transitions from the initial state in the plan.

6. The planning device according to claim 1, wherein the processor is configured to execute the instructions to search for the path from the initial state to the target state in the plan using an evaluation value for a path involving one or more state transitions, the evaluation value being calculated using evaluation values for individual state transitions, each of which is determined as a weighted sum of an evaluation value indicating, as an evaluation for a single state transition from a transition-source state to a transition-destination state, a better evaluation where the transition-destination state has higher feasibility, and an evaluation value indicating a better evaluation where the health-level-evaluation-index value in the transition-destination state shows a better evaluation relative to the health-level-evaluation-index value in the transition-source state.

7. The planning device according to claim 1, wherein the processor is configured to execute the instructions to calculate the health-level-evaluation-index value using a model that has, as parameters, a parameter that takes values following a continuous distribution and a parameter that take values following a discrete distribution have mutually different latent variables, among parameters of the health-level-evaluation-index-value.

8. The planning device according to claim 1,
wherein the state is identified using a value of one or more items correlated with a health-level-evaluation-index value and any one of a plurality of periods,
wherein the processor is configured to execute the instructions to acquire the feasibility-evaluation-index value for each value of the one or more items correlated with the health-level-evaluation-index value and for each period, and
wherein the processor is configured to execute the instructions to acquire the health-level-evaluation-index value for each value of the one or more items correlated with the health-level-evaluation-index-value and for each period.

9. The planning device according to claim 1, wherein the processor is configured to execute the instructions to calculate the health-level-evaluation-index value for each state by using a trained model that outputs, upon receiving an input of a value of one or more items identifying the state, the health-level-evaluation-index value for the defined state.

10. The planning device according to claim 1, wherein the processor is configured to execute the instructions to select one or more items used to identify the state from measurement target items for a subject of the plan, based on correlation between each measurement target item and the health-level-evaluation-index value.

11. A planning method executed by a computer, the method comprising:
acquiring, for each state identified by a value of one or more items correlated with a health-level-evaluation-index value, a feasibility evaluation index value quantitatively indicating feasibility of the state;
acquiring a health-level-evaluation-index value for each state; and
searching for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.

12. The planning method according to claim 11, wherein searching for the path comprises using the health-level-evaluation-index value in the transition-destination state as an evaluation value that indicates a better evaluation where the health-level-evaluation-index value in the transition-destination state indicates a better evaluation relative to the health-level-evaluation-index value in the transition-source state.

13. The planning method according to claim 11, further comprising
determining a target state in the plan based on the health-level-evaluation-index value for each state, and
wherein searching for the path comprises searching for a path from an initial state in the plan to the determined target state.

14. The planning method according to claim 11,
wherein searching for the path comprises searching for a path from the initial state in the plan to each state other than the initial state, and
wherein, among the detected paths, a path from the initial state to a state set as a target state is used as a path from the initial state to the target state in the plan.

15. The planning method according to claim 11, wherein acquiring the health-level-evaluation-index value comprises acquiring the health-level-evaluation-index value for each state that are reachable within a predetermined number of state transitions from the initial state in the plan.

16. The planning method according to claim 11, wherein searching for the path comprises searching for the path from the initial state to the target state in the plan using an evaluation value for a path involving one or more state transitions, the evaluation value being calculated using evaluation values for individual state transitions, each of which is determined as a weighted sum of an evaluation value indicating, as an evaluation for a single state transition from a transition-source state to a transition-destination state, a better evaluation where the transition-destination state has higher feasibility, and an evaluation value indicating a better evaluation where the health-level-evaluation-index value in the transition-destination state shows a better evaluation relative to the health-level-evaluation-index value in the transition-source state.

17. The planning method according to claim 11, wherein acquiring the health-level-evaluation-index value comprises calculating the health-level-evaluation-index value using a model that has, as parameters, a parameter that takes values following a continuous distribution and a parameter that take values following a discrete distribution have mutually different latent variables, among parameters of the health-level-evaluation-index-value.

18. The planning method according to claim 11,
wherein the state is identified using a value of one or more items correlated with a health-level-evaluation-index value and any one of a plurality of periods,
wherein acquiring the feasibility-evaluation-index value comprises acquiring the feasibility-evaluation-index value for each value of the one or more items correlated with the health-level-evaluation-index value and for each period, and
wherein acquiring the health-level-evaluation-index value comprises acquiring the health-level-evaluation-index value for each value of the one or more items correlated with the health-level-evaluation-index-value and for each period.

19. The planning method according to claim 11, wherein acquiring the health-level-evaluation-index value comprises calculating the health-level-evaluation-index value for each state by using a trained model that outputs, upon receiving an input of a value of one or more items identifying the state, the health-level-evaluation-index value for the defined state.

20. A non-transitory computer-readable recording medium that stores a program for causing a computer to execute:
acquiring, for each state identified by a value of one or more items correlated with a health-level-evaluation-index value, a feasibility evaluation index value quantitatively indicating feasibility of the state;
acquiring a health-level-evaluation-index value for each state; and
searching for a path from an initial state to a target state in a plan using an evaluation value for a path involving one or more state transitions, the evaluation value being calculated using evaluation values for individual state transitions, each of which indicates, as an evaluation for a single state transition from a transition-source state to a transition-destination state, a better evaluation where the transition-destination state has higher feasibility and also indicates a better evaluation where the health-level-evaluation-index value in the transition-destination state shows a better evaluation relative to the health-level-evaluation-index value in the transition-source state.
